(19) United States
(12) Patent Application Publication
(10) Pub. No.: US 2009/0047278 A1
(43) Pub. Date: Feb. 19, 2009

(54) Novel Combinational Use of Sulfonamide Compound

(75) Inventors: Takashi Owa, Ibaraki (JP); Yoichi Ozawa, Ibaraki (JP); Taro Semba, Ibaraki (JP); Toshiaki Wakabayashi, Ibaraki (JP)

Correspondence Address: BIRCH STEWART KOLASCH & BIRCH, PO BOX 747, FALLS CHURCH, VA, 22040-0747, US

(73) Assignee: Eisai R & D Management Co., Ltd., Tokyo (JP)

(21) Appl. No.: 11/885,081

(22) PCT Filed: Feb. 28, 2006
(86) PCT No.: PCT/JP2006/304218

§ 371 Date: Aug. 27, 2007

(30) Foreign Application Priority Data
Feb. 28, 2005 (JP) 2005-054111

(51) Int. Cl. A61K 39/395; A61K 31/404; A61K 31/18; A61K 31/343; A61K 31/498; A61K 31/5377; A61K 31/517

(52) U.S. Cl. 424/133.1; 514/415; 514/602; 514/469; 514/249; 514/234.5; 514/266.4; 424/173.1

(57) ABSTRACT

The present invention relates to a pharmaceutical composition, a kit and a method for treating cancer, comprising a sulfonamide compound in combination with a substance having an EGF inhibitory activity.

### FIELD OF THE INVENTION

The present invention relates to a novel pharmaceutical composition, a kit and a method for treating cancer, characterized by comprising a sulfonamide compound in combination with a compound having an epidermal growth factor (hereinafter, also referred to as “EGF”)) inhibitory activity, preferably an EGF receptor kinase inhibitor (hereinafter, also referred to as an “EGFR kinase inhibitor”) or an anti-EGF receptor antibody (hereinafter, also referred to as an “an anti-EGFR antibody”).

### BACKGROUND OF THE INVENTION

Examples of conventionally used chemotherapy drugs for cancer include alkylating agents such as cyclophosphamide, antimetabolites such as methotrexate and fluorouracil, antibiotics such as adriamycin, mitomycin, bleomycin, plant-derived taxol, vincristine and etoposide, and metal complexes such as cisplatin. All of them, however, have not been sufficient in anti-tumor effects, and thus there has been a strong need for development of a novel anti-tumor agent.

Recently, a sulfonamide compound has been reported as a useful anti-tumor agent⁽¹⁻⁵⁾. In particular, N-(3-chloro-1H-indole-7-yl)-4-sulfamoylbenzenesulfonamide (hereinafter, also referred to as “E7070”), N-(3-cyano-4-methyl-1H-indole-7-yl)-3-cyanobenzenesulfonamide (hereinafter, also referred to as “E7820”), N-[[(4-chlorophenyl)amino]carbonyl]-2,3-dihydro-1H-indene-5-sulfonamide (hereinafter, also referred to as “LY186641”), N-[[(3,4-dichlorophenyl)amino]carbonyl]-2,3-dihydrobenzofuran-5-sulfonamide (hereinafter, also referred to as “LY295501”), N-(2,4 dichlorobenzoyl)-4-chlorophenylsulfonamide (hereinafter, also referred to as “LY-ASAP”), N-(2,4-dichlorobenzoyl)-5-bromothiophene-2-sulfonamide (hereinafter, also referred to as “LY573636”) and 2-sulfanylamide-5-chloroquinoxaline (hereinafter, also referred to as “CQS”) are active against various types of tumors and thus are very useful.

On the other hand, as substances having an EGF inhibitory activity, EGFR kinase inhibitors 4-(3-chloro-4-fluorophenylamino)-7-methoxy-6-(3-(4-morpholino) propoxy-quinazoline) (hereinafter, also referred to as a “gefitinib”) and 4-(3-ethynylphenylamino)-6,7-bis(2-methoxyethoxy)-quinazoline (hereinafter, also referred to as “erlotinib”), and an anti-EGFR antibody cetuximab have been reported⁽⁶⁻⁹⁾.

The presence and the kind of effects resulting from combining these compounds, however, have not been reported so far.

Recently, methods were established for simultaneously detecting expression levels of multiple genes using various DNA microarrays. Thus, DNA microarrays have been used for wide-ranging purposes^{(10 and 11)}. In addition, several reports have been made about using DNA microarrays (In part, there is a macroarray using membrane filters) for examining changes in gene expressions upon use of anti-cancer drugs against tumor cells⁽¹²⁻¹⁴⁾. These reports show that the analysis of gene expression variability is highly useful in comprehensively studying the characteristic comparison among a plurality of cell populations, the biological changes in cells caused by treatment of drug or the like at molecular level.

Furthermore, reports have also been made to the analysis of gene expression profiles of 60 types of cancer cell line panels from the US National Cancer Institute for reclassification of these cell lines and examination of their characteristics⁽¹⁵⁾ and to discussion regarding relationship among the gene expression profiles of these 60 types of cancer cell line panels and sensitivity of each cell line to various anti-cancer drugs⁽¹⁶⁾.

### REFERENCES

(1) Japanese Laid-Open Patent Publication No. 7-165708.
(2) International Publication No. WO00/50395.
(3) European Patent Publication No. 0222475.
(4) International Publication No. WO02/098848.
(5) International Publication No. WO2003/035629.
(6) International Publication No. WO96/33980.
(7) Japanese Patent No. 3040486.
(8) Japanese Patent No. 3088018.
(9) Japanese Laid-Open Patent Publication No. 2-291295.
(10) Schena M, Shalon D, Davis R W, Brown P O. Science, 1995, 270, 467-70.
(11) Lockhart, D. J., Dong, H., Byrne, M. C., Follettie, M. T., Gallo, M. V., Chee, M. S., Mittmann, M., Wang C., Kobayashi, M., Horton, H. Brown, E. L., Nature Biotechnology, 1996, 14, 1675-1680.
(12) Rhee C H, Ruan S, Chen S, Chenchik A, Levin V A, Yung A W, Fuller G N, Zhang W, Oncol Rep, 1999, 6, 393-401.
(13) Zimmermann J, Erdmann D, Lalande I, Grossenbacher R, Noorani M, Furst P, Oncogene, 2000, 19, 2913-20.
(14) Kudoh K, Ramanna M, Ravatn R, Elkahloun A G, Bittner M L, Meltzer P S, Trent J M, Dalton W S, Chin K V, Cancer Res, 2000, 4161-6.
(15) Ross D T, Scherf U, Eisen M B, Perou C M, Rees C, Spellman P, Iyer V, Jeffrey S S, Van de Rijn M, Waltham M, Pergamenschikov A, Lee J C, Lashkari D, Shalon D, Myers T G, Weinstein J N, Botstein D, Brown P O, Nat Genet, 2000, 24, 227-35.
(16) Scherf U, Ross D T, Waltham M, Smith L H, Lee J K, Tanabe L, Kohn K W, Reinhold W C, Myers T G, Andrews D T, Scudiero D A, Eisen M B, Sausville E A, Pommier Y, Botstein D, Brown P O, Weinstein J N, Nat Genet, 2000, 24, 236-44.

### DISCLOSURE OF THE INVENTION

The present invention was achieved regarding the circumstances described above. The problem to be solved by the invention is to find a pharmaceutical composition and a kit having a remarkable anti-tumor activity, and a method for treating cancer.

In order to solve the above problem, the present inventors have gone through keen examination, as a result of which combinational use of E7820 and gefitinib was found to show a statistically significant (by Isobologram) synergistic antiproliferative effect in a cell proliferation assay (in vitro). In addition, combinational use of E7820 and gefitinib or erlotinib was found to show a statistically significant (by two-way ANOVA) synergistic anti-tumor effect in a subcutaneous transplant model (in vivo) of human non-small-cell lung cancer cell line. Moreover, combinational use of E7820 and gefitinib or erlotinib showed a remarkable anti-tumor effect that cannot be seen with gefitinib or erlotinib alone. The combinational use of E7820 and cetuximab was found to show a remarkable anti-tumor effect.

Furthermore, combinational use of E7070 and gefitinib or erlotinib was found to show a statistically significant (by Isobologram) synergistic antiproliferative effect.

In experiments using DNA microarrays and cancer cell line panels, genetic alteration patterns and antiproliferative activities of E7070, E7820, LY186641, LY295501, LY573636, CQS and combinations thereof were found to show high correlation. In an assay for determining antiproliferative activity, a cancer cell line resistant to E7070 was found to show cross-resistance to E7820, LY186641, LY295501, LY-ASAP, LY573636 or CQS. From these results, the present inventors have found that E7070, E7820, LY186641, LY295501, LY-ASAP, LY573636, CQS and combinations thereof have the same or similar action mechanisms that result in the same or similar gene alterations and effects.

Accordingly, E7070, E7820, LY186641, LY295501, LY-ASAP, LY573636, CQS or a combination thereof is considered to show a good anti-tumor activity when used in combination with a substance having an EGF inhibitory activity, and thus a combination of a sulfonamide compound, preferably E7070, E7820, LY186641, LY295501, LY-ASAP, LY573636, CQS or a combination thereof, and a substance having an EGF inhibitory activity, preferably gefitinib, erlotinib or cetuximab, can be used as a useful pharmaceutical composition or a kit, which can be used for treatment of cancer.

Thus, the present invention relates to:

(1) A pharmaceutical composition comprising a sulfonamide compound in combination with a substance having an EGF inhibitory activity.

(2) A kit comprising:

(a) at least one selected from the group consisting of a packaging container, an instruction and a package insert describing the combinational use of a sulfonamide compound and a substance having an EGF inhibitory activity; and

(b) a pharmaceutical composition comprising the sulfonamide compound.

(3) A kit comprising a set of a formulation comprising a sulfonamide compound and a formulation comprising a substance having an EGF inhibitory activity.

(4) Use of a sulfonamide compound for producing a pharmaceutical composition in combination with a substance having an EGF inhibitory activity.

(5) A method for treating cancer comprising administering a sulfonamide compound and a substance having an EGF inhibitory activity to a patient.

(6) A pharmaceutical composition comprising a sulfonamide compound for administering to a patient in combination with a substance having an EGF inhibitory activity.

The sulfonamide compounds according to (1)-(6) above include at least one compound selected from the group consisting of:

a compound represented by General Formula (I)

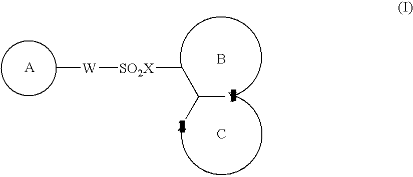

[wherein, ring A represents an optionally substituted monocyclic or bicyclic aromatic ring,

ring B represents an optionally substituted 6-membered cyclic unsaturated hydrocarbon or 6-membered unsaturated heterocycle containing a nitrogen atom as a heteroatom,

ring C represents an optionally substituted 5-membered heterocycle containing one or two nitrogen atoms,

W represents a single bond or —CH═CH—,

X represents —N(R¹)— or an oxygen atom,

Y represents

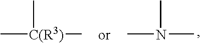

Z represents —N(R²)—,

wherein, R¹, R² and R³ independently represent, identically or differently, a hydrogen atom or a lower alkyl group];

a compound represented by General Formula (II)

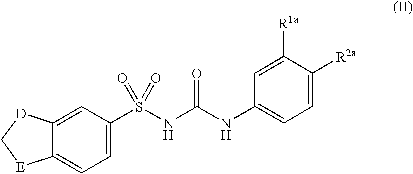

[wherein, E represents —O—, —N(CH₃)—, —CH₂—, —CH₂CH₂— or —CH₂O—, D represents —CH₂— or —O—, R^{1a} represents a hydrogen atom or a halogen atom, and R^{2a} represents a halogen atom or a trifluoromethyl group];

a compound represented by General Formula (III)

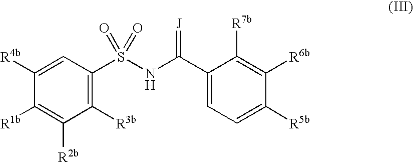

[wherein, J represents —O— or —NH—, R^{1b} represents a hydrogen atom, a halogen atom, an optionally substituted C₁-C₆ alkyl group, an optionally substituted C₁-C₄ alkoxy group, an optionally substituted C₁-C₄ alkylthio group, —CF₃, —OCF₃, —SCF₃, an optionally substituted C₁-C₄ alkoxy carbonyl group, a nitro group, an azido group, —O(SO₂)CH₃, —N(CH₃)₂, a hydroxyl group, a phenyl group, a substituted phenyl group, a pyridinyl group, a thienyl group, a furyl group, a quinolinyl group or a triazole group, R^{2b} represents a hydrogen atom, a halogen atom, a cyano group, —CF₃, an optionally substituted C₁-C₆ alkyl group, an optionally substituted C₁-C₄ alkoxy carbonyl group, an optionally substituted C₁-C₄ alkoxy group, an optionally substituted phenyl group or an optionally substituted quinolinyl group, R^{3b} represents a hydrogen atom or an optionally substituted C₁-C₄ alkoxy group, R^{4b} represents a hydrogen atom or an optionally substituted C₁-C₆ alkyl group (provided that at least one of R^{3b} and R^{4b} is a hydrogen atom), R^{5b} represents a hydrogen atom, a halogen atom, an optionally substituted C₁-C₆ alkyl group, —CF₃ or a nitro group, R^{6b} represents a hydrogen atom, a halogen atom or an optionally substituted C₁-C₆ alkyl group (provided that when R^{6b} is an optionally substituted C₁-C₆ alkyl group, R^{5b} is a hydrogen atom and R^{7b} is a halogen atom), R^{7b} represents a halogen atom, an optionally substituted C₁-C₆ alkyl group or —CF₃ (provided that when either R^{5b} or R^{7b} is an optionally substituted C₁-C₆ alkyl group or when R^{7b} is a halogen atom or an optionally substituted C₁-C₆ alkyl group, either R^{5b} or R^{6b} is a hydrogen atom)];

a compound represented by Formula (IV)

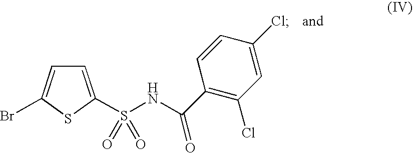

a compound represented by Formula (V)

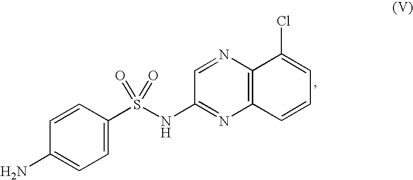

or a pharmacologically acceptable salt thereof, or a solvate thereof.

In (1)-(6) above, the substance having an EGF inhibitory activity may be an EGF receptor kinase inhibitor or an anti-EGFR antibody.

The EGF receptor kinase inhibitor may be, for example, at least one compound selected from the group consisting of:
4-(3-chloro-4-fluorophenylamino)-7-methoxy-6-(3-(4-morpholino)propoxy-quinazoline),
4-(3-ethynylphenylamino)-6,7-bis(2-methoxyethoxy)-quinazoline;
N-[3-chloro-4-[(3-fluorobenzyl)oxy]phenyl]-6-[5-[[[2-(methylsulfonyl)ethyl]amino]methyl]furan-2-yl]quinazoline-4-amine;
N-[4-[N-(3-chloro-4-fluorophenyl)amino]-7-[3-(4-morpholinyl)propoxy]quinazoline-6-yl]acrylamide;
(2E)-N-[4-[(3-chloro-4-fluorophenyl)amino]-3-cyano-7-ethoxy-6-quinolinyl]-4-(dimethylamino)-2-buteneamide;
[6-[4-[(4-ethylpiperazine-1-yl)methyl]phenyl]-7H-pyrrolo[2,3-d]pyrimidine-4-yl]-((R)-1-phenylethyl)amine; and
(E)-N-{4-[3-chloro-4-(2-pyridinylmethoxy)anilino]-3-cyano-7-ethoxy-6-quinolinyl}-4-(dimethylamino)-2-buteneamide,
   or a pharmacologically acceptable salt thereof or a solvate thereof.

The anti-EGFR antibody is, for example, at least one antibody selected from the group consisting of cetuximab, panitumumab, matuzumab, nimotuzumab, IMC-11F8 and MDX-447.

The present invention provides a pharmaceutical composition and a kit that show a remarkable anti-tumor activity, and a method for treating cancer.

More specifically, the present invention provides a pharmaceutical composition and a kit that show a remarkable anti-tumor activity and a method for treating cancer, by combining a sulfonamide compound, that is, at least one compound selected from (A) a compound represented by General Formula (I), preferably E7070 or E7820, (B) a compound represented by General Formula (II), preferably LY186641 or LY295501, (C) a compound represented by General Formula (III), preferably LY-ASAP, (D) LY573636 and (E) CQS, with a substance having an EGF inhibitory activity, preferably at least one selected from gefitinib, erlotinib and cetuximab. Thus, the pharmaceutical composition, the kit and the method of the invention can be used for cancer treatment.

### BRIEF DESCRIPTION OF THE DRAWINGS

FIG. 1 is a theoretical view of Isobologram method.

FIG. 2 shows the effect of combinational use of E7820 and gefitinib in a cell proliferation assay according to Isobologram method.

FIG. 3 shows the effect of combinational use of E7070 and gefitinib in a cell proliferation assay according to Isobologram method.

FIG. 4 shows the effect obtained by combinational use of E7070 and erlotinib in a cell proliferation assay according to Isobologram method.

FIG. 5 shows the effect obtained by combinational use of E7820 and gefitinib in a subcutaneous transplant model of human non-small-cell lung cancer cell line (PC9). In the figure, * indicates a statistically significant synergistic effect at a significance level of less than 0.01. In the figure, Day# indicates days from the first day of administration (Day 1).

FIG. 6 shows the effect obtained by combinational use of E7820 and gefitinib in a subcutaneous transplant model of human non-small-cell lung cancer cell line (A549). In the figure, * indicates a statistically significant synergistic effect at a significance level of less than 0.01. In the figure, Day# indicates days from the first day of administration (Day 1).

FIG. 7 shows the effect obtained by combinational use of E7820 and erlotinib in a subcutaneous transplant model of human non-small-cell lung cancer cell line (A549). In the figure, * indicates a statistically significant synergistic effect at a significance level of less than 0.01. In the figure, Day# indicates days from the first day of administration (Day 1).

FIG. 8 shows the results of hierarchical cluster analysis in the DNA microarrays in Example 7.

FIG. 9 shows correlation coefficients in the DNA microarrays in Example 8.

FIG. 10 shows the results of hierarchical cluster analysis in the DNA microarrays in Example 8.

FIG. 11 shows correlation coefficients in the DNA microarrays in Example 8.

FIG. 12 shows the results of hierarchical cluster analysis in the DNA microarrays in Example 8.

FIG. 13 shows antiproliferative effects of E7070, E7820, CQS, LY186641, LY295501 and LY-ASAP on HCT116-C9, HCT116-C9-C1 and HCT116-C₉-C₄ as measured by cell growth inhibition assay.

FIG. 14 shows antiproliferative effects of E7070 and LY573636 on HCT116-C9, HCT116-C9-C1 and HCT116-C₉-C₄ as measured by cell growth inhibition assay.

### BEST MODES FOR CARRYING OUT THE INVENTION

Hereinafter, embodiments of the present invention will be described. The following embodiments are described for illustrating the present invention and they are not intended to limit the present invention. The present invention may be carried out in various embodiments as long as it does not depart from the scope of the invention.

The publications, laid-open patent publications, patent publications and other patent documents cited herein are incorporated herein by reference.

1. Sulfonamide Compound

A pharmaceutical composition and/or a kit and a method for treating cancer of the present invention comprise a sulfonamide compound.

According to the present invention, the sulfonamide compound comprises a compound represented by the following General Formula (I).

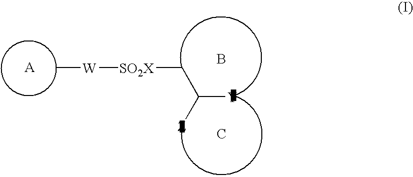

In General Formula (I),

ring A represents an optionally substituted monocyclic or bicyclic aromatic ring,

ring B represents an optionally substituted 6-membered cyclic unsaturated hydrocarbon or 6-membered unsaturated heterocycle containing a nitrogen atom as a heteroatom,

ring C represents an optionally substituted 5-membered heterocycle containing one or two nitrogen atoms,

W represents a single bond or —CH═CH—,

X represents —N(R¹)— or an oxygen atom,

Y represents

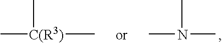

Z represents —N(R²)—.

R¹, R² and R³ independently represent, identically or differently, a hydrogen atom or a lower alkyl group.

In General Formula (I), “an optionally substituted monocyclic or bicyclic aromatic ring” meant by ring A is an aromatic hydrocarbon or an aromatic heterocycle containing at least one of a nitrogen atom, an oxygen atom and a sulfur atom, which may have 1 to 3 substituents on the ring. Examples of the aromatic ring comprised in ring A mainly include pyrrole, pyrazole, imidazole, thiophene, furan, thiazole, oxazole, benzene, pyridine, pyrimidine, pyrazine, pyridazine, naphthalene, quinoline, isoquinoline, phthalazine, naphthyridine, quinoxaline, quinazoline, cinnoline, indole, isoindole, indolizine, indazole, benzofuran, benzothiophene, benzoxazole, benzimidazole, benzopyrazole and benzothiazole, although the aromatic ring comprised in ring A is not limited thereto. The aromatic ring may have 1 to 3 substituents, and when more than one substituent exist, they may be identical or different. Examples of the substituent include an amino group that may be substituted with a lower alkyl group or a lower cyclo alkyl group, a lower alkyl group, a lower alkoxy group, a hydroxyl group, a nitro group, a mercapto group, a cyano group, a lower alkylthio group, a halogen atom, a group represented by Formula -a-b [wherein, a represents a single bond, —(CH₂)ₖ—, —O—(CH₂)ₖ—, —S—(CH₂)ₖ— or —N(R³)—(CH₂)ₖ—, k represents an integer of 1-5, R³ refers to a hydrogen atom or a lower alkyl group, b represents —CH₂-d (wherein, d represents an amino group that may be substituted with a lower alkyl group, a halogen atom, a hydroxyl group, a lower alkylthio group, a cyano group or a lower alkoxy group)], a group represented by Formula -a-e-f [wherein, a is as stated above, e represents —S(O)— or —S(O)₂—, f represents an amino group that may be substituted with a lower alkyl group or a lower alkoxy group, a lower alkyl group, a trifluoromethyl group, —(CH₂)ₘ-b or —N(R⁴)—(CH₂)ₘ-b (wherein, b is as stated above, R⁴ represents a hydrogen atom or a lower alkyl group, m represents an integer of 1-5)], a group represented by Formula -a-g-h [wherein, a is as stated above, g represents —C(O)— or —C(S)—, h represents an amino group that may be substituted with a lower alkyl group, a hydroxyl group, a lower alkyl group, a lower alkoxy group, —(CH₂)ₙ-b or —N(R⁵)—(CH₂)ₙ-b (wherein, b is as stated above, R⁵ represents a hydrogen atom or a lower alkyl group, and n represents an integer of 1-5)], a group represented by Formula -a-N(R⁶)-g-i [wherein, a and g are as stated above, R⁶ represents a hydrogen atom or a lower alkyl group, and i represents a hydrogen atom, a lower alkoxy group or f (f is as stated above)], a group represented by Formula -a-N(R⁷)-e-f (wherein, a, e and f are as stated above, R⁷ refers to a hydrogen atom or a lower alkyl group), and a group represented by Formula —(CH₂)ₚ-j-(CH₂)_{q}-b (wherein, j represents an oxygen atom or a sulfur atom, b is as stated above, p and q identically or differently represent an integer of 1-5).

Among the exemplary substituents mentioned above, when the amino group is substituted with two alkyl groups, these alkyl groups may bind to each other to form a 5 or 6-membered ring. When ring A is a nitrogen-containing heterocycle having a hydroxyl group or a mercapto group, these groups may take a resonance structure and form an oxo group or a thioxo group.

In General Formula (I), “an optionally substituted 6-membered cyclic unsaturated hydrocarbon or 6-membered unsaturated heterocycle containing a nitrogen atom as a heteroatom” meant by ring B, for example, is benzene or pyridine in which a part of the unsaturated binding may be hydrogenated, which may have one or two substituents on the ring. When two or more substituents exist, they may be identical or different.

“An optionally substituted 5-membered heterocycle containing one or two nitrogen atoms” meant by ring C is pyrrole, pyrazole or imidazole in which a part of the unsaturated binding may be hydrogenated, which may have one or two substituents on the ring. When two or more substituents exist, they may be identical or different.

In General Formula (I), Z represents —N(R²)—. R² and R¹ independently represent, identically or differently, a hydrogen atom or a lower alkyl group.

Examples of substituents that rings B and C may have include but not limited to a halogen atom, a cyano group, a lower alkyl group, a lower alkoxy group, a hydroxyl group, an oxo group, Formula —C(O)-r (wherein, r represents a hydrogen atom, an amino group that may be substituted with a lower alkyl group, a lower alkyl group, a lower alkoxy group or a hydroxyl group), an amino group that may be substituted with a lower alkyl group and a trifluoromethyl group.

In General Formula (I), Y represents

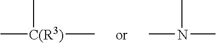

(wherein R³ represents a hydrogen atom or a lower alkyl group).

In General Formula (I), “lower alkyl group” in the definition of the substituents that R¹, R², R³, ring A, ring B and ring C may have refers to a linear or branched alkyl group with a carbon number of 1-6, for example, but not limited to, a methyl group, an ethyl group, a n-propyl group, an isopropyl group, a n-butyl group, an isobutyl group, a sec-butyl group, a tert-butyl group, a n-pentyl group (an amyl group), an isopentyl group, a neopentyl group, a tert-pentyl group, a 1-methylbutyl group, a 2-methylbutyl group, a 1,2-dimethylpropyl group, a n-hexyl group, an isohexyl group, a 1-methylpentyl group, a 2-methylpentyl group, a 3-methylpentyl group, a 1-ethylpropyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,1,2-trimethylpropyl group, a 1,2,2-trimethylpropyl group, a 1-ethyl-1-methylpropyl group and a 1-ethyl-2-methylpropyl group. Among these, examples of preferable groups include a methyl group, an ethyl group, a n-propyl group, an isopropyl group, a n-butyl group, an isobutyl group, a sec-butyl group and a tert-butyl group, while examples of the most preferable groups include a methyl group, an ethyl group, a n-propyl group and an isopropyl group.

The “lower cyclo alkyl group” in the definition of the substituent that ring A may have refers to a cyclo alkyl group with a carbon number of 3-8, for example, but not limited to, a cyclopropyl group, a cyclobutyl group, a cyclopentyl group, a cyclohexyl group, a cycloheptyl group and a cyclooctyl group. The “lower alkylthio group” also refers to an alkylthio group derived from the lower alkyl group, for example, but not limited to, a methylthio group, an ethylthio group, a n-propylthio group, an isopropylthio group, a n-butylthio group, an isobutylthio group, a sec-butylthio group and a tert-butylthio group.

The “lower alkoxy group” in the definition of the substituents that ring A, ring B and ring C may have, for example, refers to, but not limited to, a lower alkoxy group derived from a lower alkyl group such as a methoxy group, an ethoxy group, a n-propoxy group, an isopropoxy group, a n-butoxy group, an isobutoxy group, a sec-butoxy group and a tert-butoxy group, the most preferable group being a methoxy group and an ethoxy group. In addition, examples of a “halogen atom” include a fluorine atom, a chlorine atom, a bromine atom and an iodine atom.

The compound represented by General Formula (I) of the invention can be produced according to a known method, for example, by those described in International Publication No. 95/07276 (pamphlet) (WO95/07276) and/or Japanese Laid-Open Patent Publication No. 7-165708 (JP7-165708).

In General Formula (I), a preferable compound is E7070 or E7820.

E7070 is N-(3-chloro-1H-indole-7-yl)-4-sulfamoylbenzenesulfonamide, whose structural formula is represented by the following Formula (VIII).

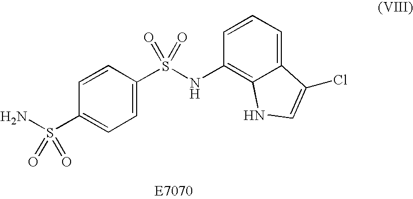

E7070 can be produced according to a known method, for example, by those described in International Publication No. 95/07276 (pamphlet) (WO95/07276) and/or Example 19 of Japanese Laid-Open Patent Publication No. 7-165708 (JP7-165708).

E7820 is N-(3-cyano-4-methyl-1H-indole-7-yl)-3-cyanobenzenesulfonamide, whose structural formula is represented by the following Formula (IX).

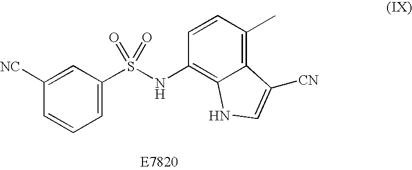

E7820 can be produced according to a known method, for example, by a method described in International Publication No. 00/50395 (pamphlet) (WO00/50395).

According to the present invention, the sulfonamide compound comprises a compound represented by the following General Formula (II).

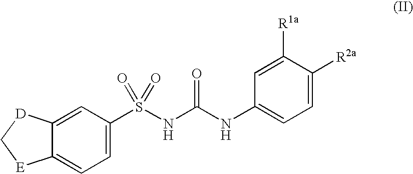

In General Formula (II) above, E represents —O—, —N(CH₃)—, —CH₂—, —CH₂CH₂— or —CH₂O—, D represents —CH₂— or —O—, R^{1a} represents a hydrogen atom or a halogen atom (e.g., a fluorine atom, a chlorine atom, a bromine atom or an iodine atom), and R^{2a} represents a halogen atom or a trifluoromethyl group.

The compound represented by General Formula (II) of the invention can be produced according to a known method, for example, by a method described in European Patent Publication No. 0222475A1 (specification) (EP0222475A1).

In General Formula (II), a preferable compound is LY186641 or LY295501.

LY186641 is N-[[(4-chlorophenyl)amino]carbonyl]-2,3-dihydro-1H-indene-5-sulfonamide, whose structural formula is represented by the following Formula (X).

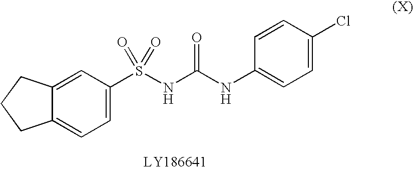

LY186641 can be produced according to a known method, for example, by a method described in European Patent Publication No. 0222475A1 (specification) (EP0222475A1).

According to the present invention, LY295501 is N-[[(3,4-dichlorophenyl)amino]carbonyl]-2,3-dihydrobenzofuran-5-sulfonamide, whose structural formula is represented by the following Formula (XI).

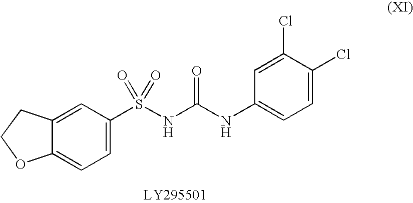

LY295501 can be produced according to a known method, for example, by those described in European Patent Publication No. 0222475A1 (specification) (EP0222475A1) and/or European Patent Publication No. 0555036A2 (specification) (EP0555036A2).

Furthermore, according to the present invention, the sulfonamide compound comprises a compound represented by the following General Formula (III).

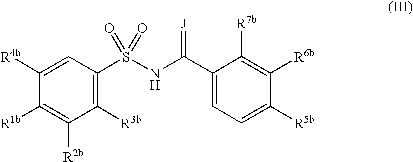

In General Formula (III), J represents —O— or —NH—, R^{1b} represents a hydrogen atom, a halogen atom, an optionally substituted C₁-C₆ alkyl group, an optionally substituted C₁-C₄ alkoxy group, an optionally substituted C₁-C₄ alkylthio group, —CF₃, —OCF₃, —SCF₃, an optionally substituted C₁-C₄ alkoxy carbonyl group, a nitro group, an azido group, —O(SO₂)CH₃, —N(CH₃)₂, a hydroxyl group, a phenyl group, a substituted phenyl group, a pyridinyl group, a thienyl group, a furyl group, a quinolinyl group or a triazole group, R^{2b} represents a hydrogen atom, a halogen atom, a cyano group, —CF₃, an optionally substituted C₁-C₆ alkyl group, an optionally substituted C₁-C₄ alkoxy carbonyl group, an optionally substituted C₁-C₄ alkoxy group, an optionally substituted phenyl group or an optionally substituted quinolinyl group, R^{3b} represents a hydrogen atom or an optionally substituted C₁-C₄ alkoxy group, R^{4b} represents a hydrogen atom or an optionally substituted C₁-C₆ alkyl group (provided that at least one of R^{3b} and R^{4b} is a hydrogen atom), R^{5b} refers to a hydrogen atom, a halogen atom, an optionally substituted C₁-C₆ alkyl group, —CF₃ or a nitro group, R^{6b} refers to a hydrogen atom, a halogen atom or an optionally substituted C₁-C₆ alkyl group (provided that when R^{6b} is an optionally substituted C₁-C₆ alkyl group, R^{5b} is a hydrogen atom and R^{7b} is a halogen atom), R^{7b} refers to a halogen atom, an optionally substituted C₁-C₆ alkyl group or —CF₃ (provided that when either R^{5b} or R^{7b} is an optionally substituted C₁-C₆ alkyl group or when R^{7b} is a halogen atom or an optionally substituted C₁-C₆ alkyl group, either R^{5b} or R^{6b} is a hydrogen atom).

In General Formula (III), a “halogen atom” is preferably a fluorine atom, a chlorine atom, a bromine atom or an iodine atom.

In General Formula (III), “C₁-C₆ alkyl group” is synonymous with the “lower alkyl group” described above, and preferably includes, but not limited to, a methyl group, an ethyl group, a n-propyl group, an isopropyl group, a n-butyl group, an isobutyl group, a sec-butyl group, a tert-butyl group, a n-pentyl group and a n-hexyl group.

In General Formula (III), “C₁-C₄ alkoxy group” refers to an alkoxy group with a carbon number of 1-4 of the “lower alkoxy groups” described above, and preferably includes, but not limited to, a methoxy group, an ethoxy group, a n-propoxy group, an isopropoxy group, a n-butoxy group, an isobutoxy group, a sec-butoxy group and a tert-butoxy group.

In General Formula (III), examples of alkyl group of “C₁-C₄ alkylthio group” include, but not limited to, methyl, ethyl, n-propyl, isopropyl, n-butyl, isobutyl, sec-butyl and tert-butyl.

In General Formula (III), examples of “C₁-C₄ alkoxy carbonyl group” include, but not limited to, a methoxy carbonyl group, an ethoxy carbonyl group, a n-propoxy carbonyl group, an isopropoxy carbonyl group, a n-butoxy carbonyl group, an isobutoxy carbonyl group, a sec-butoxy carbonyl group and a tert-butoxy carbonyl group.

In General Formula (III), examples of substituents to be introduced include, but not limited to, substituents such as a C₁-C₆ alkyl group (e.g., a methyl group, an ethyl group, a n-propyl group, an isopropyl group, a n-butyl group, an isobutyl group, a sec-butyl group, a tert-butyl group, etc.), a C₁-C₄ alkoxy group (e.g., a methoxy group, an ethoxy group, a n-propoxy group, an isopropoxy group, a n-butoxy group, an isobutoxy group, a sec-butoxy group, a tert-butoxy group, etc.), an amino group, a hydroxyl group, a halogen atom (e.g., a fluorine atom, a chlorine atom, a bromine atom or an iodine atom) and a silyl group.

The compound represented by General Formula (III) of the invention can be produced by a known method such as the method described in International Publication No. 02/098848 (pamphlet) (WO02/098848).

In General Formula (III), a preferable compound is LY-ASAP.

LY-ASAP is N-(2,4-dichlorobenzoyl)-4-chlorophenylsulfonamide, whose structural formula is represented by the following Formula (XII).

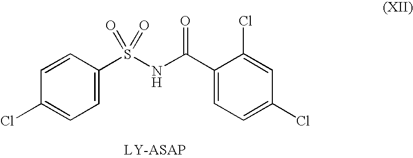

LY-ASAP can be produced by a known method such as the method described in International Publication No. 02/098848 (pamphlet) (WO02/098848).

According to the present invention, an example of the sulfonamide compound includes LY573636. According to the invention, LY573636 is N-(2,4-dichlorobenzoyl)-5-bromothiophene-2-sulfonamide, whose structural formula is represented by the following Formula (IV).

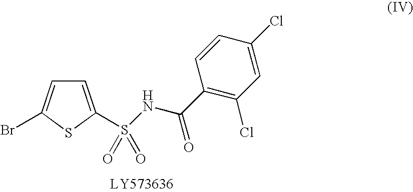

LY573636 is preferably in sodium salt form.

LY573636 can be produced by a known method. For example, it can be produced in the same manner as the method described in International Publication No. 02/098848 (pamphlet) (WO02/098848) using commercially available 5-bromothiophene-2-sulfonyl chloride and 2,4-dichlorobenzoic acid.

LY573636 can be produced by a method described in Example 63 of International Publication No. 2003/035629 (pamphlet) (WO2003/035629).

According to the present invention, the sulfonamide compound may be CQS. According to the present invention, CQS is 2-sulfanylamide-5-chloroquinoxaline, whose structural formula is represented by the following Formula (V).

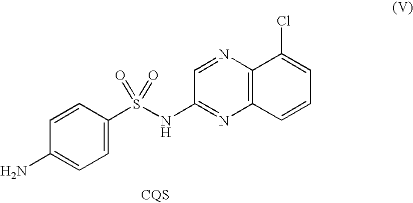

CQS can be produced according to a known method, for example, by a method described in J. Am. Chem. Soc., 1947, 71, 6-10.

The sulfonamide compound may form a pharmacologically acceptable salt with acid or base. The sulfonamide compound of the invention also comprises these pharmacologically acceptable salts. Examples of salts formed with acids include inorganic acid salts such as hydrochloride salts, hydrobromide salts, sulfate salts and phosphate salts, and salts formed with organic acids such as formic acid, acetic acid, lactic acid, succinic acid, fumaric acid, maleic acid, citric acid, tartaric acid, benzoic acid, methanesulfonic acid, benzenesulfonic acid, p-toluenesulfonic acid and trifluoroacetic acid. Examples of salts formed with bases include alkali metal salts such as sodium salt and potassium salt, alkaline earth metal salts such as calcium salt and magnesium salt, salts with organic bases such as trimethylamine, triethylamine, pyridine, picoline, dicyclohexylamine, N,N′-dibenzylethylenediamine, arginine and lysine (organic amine salts), and ammonium salts.

Furthermore, the sulfonamide compound may be in anhydride form, and may form a solvate such as a hydrate. The solvate may be either a hydrate or a nonhydrate, preferably a hydrate. The solvent used may be water, alcohol (e.g., methanol, ethanol or n-propanol), dimethylformamide or the like.

If solvates and/or enantiomers of these compounds exist, the sulfonamide compound of the invention comprises these solvates and/or enantiomers. The sulfonamide compound of the invention also comprises a sulfonamide compound that undergoes metabolism such as oxidation, reduction, hydrolysis and conjugation in vivo. Moreover, the sulfonamide compound of the invention also comprises compounds that generate a sulfonamide compound by undergoing metabolism such as oxidation, reduction and hydrolysis in vivo.

2. Substance Having EGF Inhibitory Activity

A pharmaceutical composition and/or a kit and a method of the invention for the treatment of cancer comprise a substance having an EGF inhibitory activity. According to the present invention, the substance having an EGF inhibitory activity is not particularly limited as long as it inhibits EGF action, activity or the like, but preferably is an EGF Receptor (EGFR) kinase inhibitor or an anti-EGF Receptor (EGFR) antibody.

EGF inhibitory activity refers to activity in inhibition of physiological activity and/or pharmacological activity of EGF. EGF inhibitory activity may be determined by an existing method, for example, cell proliferation assay, kinase assay or western blotting. The substance may be assumed to be a substance having EGF inhibitory activity when an EGF inhibitory activity quantified by these methods is, for example, 30 μM or lower, preferably 10 μM or lower, more preferably 3 μM or lower, still more preferably 1 μM or lower at 50% inhibitory concentration.

(1) EGFR Kinase Inhibitor

According to the present invention, an EGFR kinase inhibitor may comprise a compound represented by General Formula (VI).

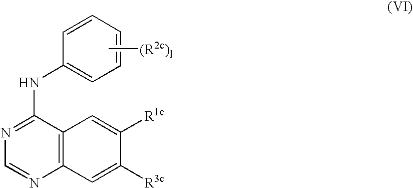

wherein, l represents 1, 2 or 3,

R^{2c} independently represents a halogen atom, a trifluoromethyl group or a C₁-C₄ alkyl group,

R^{3c} represents a C₁-C₄ alkoxy group,

R^{1c} represents a di-[(C₁-C₄)alkyl]amino-(C₂-C₄)alkoxy group,

a pyrrolidine-1-yl-(C₂-C₄)alkoxy group,

a piperidino-(C₂-C₄)alkoxy group,

a morpholino-(C₂-C₄)alkoxy group,

a piperazine-1-yl-(C₂-C₄)alkoxy group,

a 4-(C₁-C₄)alkylpiperazine-1-yl-(C₂-C₄)alkoxy group,

an imidazole-1-yl-(C₂-C₄)alkoxy group,

a di-[(C₁-C₄)alkoxy-(C₂-C₄)alkyl]amino-(C₂-C₄)alkoxy group,

a thiamorpholino-(C₂-C₄)alkoxy group,

a 1-oxothiamorpholino-(C₂-C₄)alkoxy group

or

a 1,1-dioxothiamorpholino-(C₂-C₄)alkoxy group

(provided that when R^{1c} has —CH₂— (a methylene group) that is not attached to N or O atom, any one or more of the methylene groups may have a hydroxyl group on the carbon atom).

In General Formula (VI), “C₁-C₄ alkyl group” refers to a linear or branched alkyl group with a carbon number of 1-4 of the “lower alkyl groups” described above.

In General Formula (VI), “C₁-C₄ alkoxy group” is synonymous with the “C₁-C₄ alkoxy group” described above.

In General Formula (VI), “C₂-C₄ alkoxy group” refers to a alkoxy group with a carbon number of 2-4 of the “lower alkoxy groups” described above.

In General Formula (VI), R^{2c} is preferably, but not limited to, a halogen atom or a C₁-C₄ alkyl group. If R^{2c} is a halogen atom, it is, for example, a fluorine atom, a chlorine atom, a bromine atom or an iodine atom, and if R^{2c} is a C₁-C₄ alkyl group, it is, for example, a methyl group, an ethyl group, a propyl group, an isopropyl group or a butyl group.

In General Formula (VI), examples of R^{3c} preferably include, but not limited to, a methoxy group, an ethoxy group, a propoxy group, an isopropoxy group or a butoxy group.

In General Formula (VI), R^{1c} is preferably, but not limited to:

a di-[(C₁-C₄)alkyl]amino-(C₂-C₄)alkoxy group such as a 2-dimethylaminoethoxy group, a 2-(N-ethyl-N-methylamino)ethoxy group, a 2-diethylaminoethoxy group, a 2-dipropylaminoethoxy group, a 3-dimethylaminopropoxy group, a 3-diethylaminopropoxy group, a 2-dimethylaminopropoxy group, a 2-diethylaminopropoxy group, a 1-dimethylaminopropane-2-yloxy group, a 1-diethylaminopropane-2-yloxy group, a 1-dimethylamino-2-methylpropane-2-yloxy group, a 2-dimethylamino-2-methylpropoxy group, a 4-dimethylaminobutoxy group, a 4-diethylaminobutoxy group, a 3-dimethylaminobutoxy group, a 3-diethylaminobutoxy group, a 2-dimethylaminobutoxy group, a 2-diethylaminobutoxy group, a 1-dimethylaminobutane-2-yloxy group or a 1-diethylaminobutane-2-yloxy group;

a pyrrolidine-1-yl-(C₂-C₄)alkoxy group such as a 2-(pyrrolidine-1-yl)ethoxy group, a 3-(pyrrolidine-1-yl)propoxy group or a 4-(pyrrolidine-1-yl)butoxy group;

a piperidino-(C₂-C₄)alkoxy group such as a 2-piperidinoethoxy group, a 2-piperidinopropoxy group, a 3-piperidinopropoxy group or a 4-piperidinobutoxy group;

a morpholino-(C₂-C₄)alkoxy group such as a 2-morpholinoethoxy group, a 3-morpholinopropoxy group or a 4-morpholinobutoxy group;

a piperazine-1-yl-(C₂-C₄)alkoxy group such as a 2-(piperazine-1-yl)ethoxy group, a 3-(piperazine-1-yl)propoxy group or a 4-(piperazine-1-yl)butoxy group;

a 4-(C₁-C₄)alkylpiperazine-1-yl-(C₂-C₄)alkoxy group such as a 2-(4-methylpiperazine-1-yl)ethoxy group, a 3-(4-methylpiperazine-1-yl)propoxy group or a 4-(4-methylpiperazine-1-yl)butoxy group;

an imidazole-1-yl-(C₂-C₄)alkoxy group such as a 2-(imidazole-1-yl)ethoxy group, a 3-(imidazole-1-yl)propoxy group or a 4-(imidazole-1-yl)butoxy group;

a di-[(C₁-C₄)alkoxy-(C₂-C₄)alkyl]amino-(C₂-C₄)alkoxy group such as a 2-[di-(2-methoxyethyl)amino]ethoxy group, a 3-[di-(2-methoxyethyl)amino]propoxy group, a 2-[di-(3-methoxypropyl)amino]ethoxy group or a 3-[di-(3-methoxypropyl)amino]propoxy group;

a thiamorpholino-(C₂-C₄)alkoxy group such as a 2-thiamorpholinoethoxy group, a 3-thiamorpholinopropoxy group or a 4-thiamorpholinobutoxy group;

a 1-oxothiamorpholino-(C₂-C₄)alkoxy group such as a 2-(1-oxothiamorpholino)ethoxy group, a 3-(1-oxothiamorpholino)propoxy group or a 4-(1-oxothiamorpholino)butoxy group; or

a 1,1-dioxothiamorpholino-(C₂-C₄)alkoxy group such as a 2-(1,1-dioxothiamorpholino)ethoxy group, a 3-(1,1-dioxothiamorpholino)propoxy group or a 4-(1,1-dioxothiamorpholino)butoxy group.

When R^{1c} has —CH₂— (a methylene group) that is not attached to N or O atom, R^{1c} is preferably, but not limited to, a morpholino-(C₂-C₄)alkoxy group or a di-[(C₁-C₄)alkyl]amino-(C₂-C₄)alkoxy group in which carbon atoms of any one or more of the methylene groups above are substituted with hydroxyl groups, for example, a hydroxy-morpholino-(C₂-C₄)alkoxy group, or a hydroxy-di-[(C₁-C₄)alkyl]amino-(C₂-C₄)alkoxy group such as a 2-hydroxy-3-morpholinopropoxy group or a 3-dimethylamino-2-hydroxypropoxy group.

In General Formula (VI), more preferable but non-limiting compounds comprise
4-(3′-chloro-4′-fluoroanilino)-7-methoxy-6-(2-pyrrolidine-1-ylethoxy)quinazoline,
4-(3′-chloro-4′-fluoroanilino)-7-methoxy-6-(2-morpholinoethoxy)quinazoline,
4-(3′-chloro-4′-fluoroanilino)-7-methoxy-6-[2-(4-methylpiperazine-1-yl)ethoxy]quinazoline,
4-(3′-chloro-4′-fluoroanilino)-7-methoxy-6-{2-[di-(2-methoxyethyl)amino]ethoxy}-quinazoline,
4-(3′-chloro-4′-fluoroanilino)-6-(2-dimethylaminoethoxy)-7-methoxyquinazoline,
4-(3′-chloro-4′-fluoroanilino)-6-(2-diethylaminoethoxy)-7-methoxyquinazoline,
4-(2′,4′-difluoroanilino)-6-(3-dimethylaminopropoxy)-7-methoxyquinazoline,
4-(3′-chloro-4′-fluoroanilino)-6-(2-hydroxy-3-morpholinopropoxy)-7-methoxyquinazoline,
4-(2′,4′-difluoroanilino)-7-methoxy-6-(3-morpholinopropoxy)quinazoline,
4-(3′-chloro-4′-fluoroanilino)-6-(2-imidazole-1-ylethoxy)-7-methoxyquinazoline,
4-(3′-chloro-4′-fluoroanilino)-6-(3-diethylaminopropoxy)-7-methoxyquinazoline,
4-(3′-chloro-4′-fluoroanilino)-7-methoxy-6-(3-pyrrolidine-1-ylpropoxy)quinazoline,
4-(3′-chloro-4′-fluoroanilino)-6-(3-dimethylaminopropoxy)-7-methoxyquinazoline,
4-(3′,4′-difluoroanilino)-6-(3-dimethylaminopropoxy)-7-methoxyquinazoline,
4-(3′,4′-difluoroanilino)-7-methoxy-6-(3-morpholinopropoxy)-quinazoline,
6-(3-diethylaminopropoxy)-4-(3′,4′-difluoroanilino)-7-methoxyquinazoline,
4-(3′-chloro-4′-fluoroanilino)-7-methoxy-6-(3-piperidinopropoxy)quinazoline,
4-(3′-chloro-4′-fluoroanilino)-7-methoxy-6-(2-piperidinopropoxy)quinazoline,
4-(3′-chloro-4′-fluoroanilino)-6-(3-imidazole-1-ylpropoxy)-7-methoxyquinazoline, and
4-(3-chloro-4-fluorophenylamino)-7-methoxy-6-(3-(4-morpholino)propoxy-quinazoline).

A compound represented by General Formula (VI) can be produced by a known method, for example, those that are described in International Publication No. 96/33980 (pamphlet) (WO96/33980), Japanese Patent No. 3040486 (JP3040486) and U.S. Pat. No. 5,770,599 (specification) (U.S. Pat. No. 5,770,599).

In General Formula (VI), a particularly preferable compound is gefitinib.

Gefitinib is 4-(3-chloro-4-fluorophenylamino)-7-methoxy-6-(3-(4-morpholino)propoxy-quinazoline), whose structural formula is represented by the following Formula (XIII).

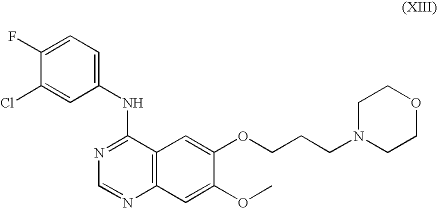

Gefitinib can be produced according to a known method, for example, by those described in International Publication No. 96/33980 (pamphlet) (WO96/33980), Japanese Patent No. 3040486 (JP3040486) and U.S. Pat. No. 5,770,599 (specification) (U.S. Pat. No. 5,770,599).

Also, gefitinib can be obtained by purchasing Iressa® from AstraZeneca.

An example of the EGFR kinase inhibitor includes a compound represented by General Formula (VII).

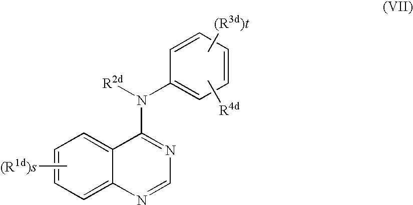

In General Formula (VII), s represents 1, 2 or 3.

In General Formula (VII), R^{1d} may be (a) or (b) below.

(a) Each R^{1d} is independently selected from a hydrogen atom, a halogen atom, a hydroxyl group, an amino group, a hydroxyamino group, a carboxy group, a C₁-C₄ alkoxy carbonyl group, a nitro group, a guanidino group, an ureide group, a carbamoyl group, a cyano group, a trifluoromethyl group, a (R^{6d})₂N-carbonyl group and a phenyl-U—(C₁-C₄) alkyl group (wherein, U is selected from a single bond, O, S and NH).

(b) Each R^{1d} is independently selected from a cyano-(C₁-C₄) alkyl group, a R^{5d}-sulfonylamino group, a phthalimide-(C₁-C₄)alkylsulfonylamino group, a benzamide group, a benzenesulfonylamino group, a 3-phenylureide group, a 2-oxopyrrolidine-1-yl group, a 2,5-dioxopyrrolidine-1-yl group, a R^{10d}—(C₂-C₄)alkanoyl amino group and R^{9d}.

In these cases, R^{5d} is a C₁-C₄ alkyl group;

R^{6d} is a hydrogen atom or a C₁-C₄ alkyl group;

R^{9d} is selected from a C₁-C₄ alkyl group, a C₁-C₄ alkoxy group, (R^{6d})₂N— (where R^{6d} may be identical or different), (R^{6d})₂NC(═O)— (where R^{6d} may be identical or different), R^{7d}C(═O)— (where R^{7d} may be identical or different), R^{5d}ONH— (where R^{5d} may be identical or different), R^{5d}NH— (where R^{5d} may be identical or different), R^{5d}NHC(═O)— (where R^{5d} may be identical or different), (R^{5d})₂NC(═O)— (where R^{5d} may be identical or different), G and R^{5d}V (where R^{5d} may be identical or different);

R^{10d} is selected from a halogen atom, a hydroxyl group, a carboxy group, a carbamoyl group, an N—(C₁-C₄)alkylcarbamoyl group, an N,N-di-(C₁-C₄)alkylcarbamoyl group, a C₁-C₄ alkylamino group, a C₁-C₄ alkoxy group, R^{6d}O— (where R^{6d} may be identical or different), a C₂-C₄ alkanoyloxy group, R^{7d}C(═O)— (where R^{7d} may be identical or different), and (R^{6d})₂N— (where R^{6d} may be identical or different);

R^{7d} is a C₁-C₄ alkyl group, a C₁-C₄ alkoxy group or (R^{6d})₂N— (where R^{6d} may be identical or different);

G is selected from a piperidino group, a morpholino group, a pyrrolidino group, a 4-R^{6d}-piperidine-1-yl group (where R^{6d} may be identical or different), an imidazole-1-yl group, a 4-pyridone-1-yl group, a carboxy-(C₁-C₄) alkyl group, a phenoxy group, a phenyl group, a C₁-C₄ alkylsulfanyl group, a phenylsulfanyl group, a C₂-C₄ alkenyl group, an anilino group and a (R^{6d})₂N-carbonyl-(C₁-C₄) alkyl group (where R^{6d} may be identical or different); and

V is selected from S—, SO— and SO₂—.

In General Formula (VII), each of R^{1d} may cross-link with each other to form a C₄-C₈ saturated or unsaturated ring. In addition, each of R^{3d} or each of R^{3d} and R^{4d} may cross-link with each other to form a C₄-C₈ saturated or unsaturated ring. A ring formed with these substituents is preferably a 4-8-membered ring, and more preferably 4-7-membered ring. This ring may be an aromatic ring such as a benzene ring, or an aliphatic ring. Moreover, in addition to a ring formed with these substituents, one or more additional rings may be formed.

In General Formula (VII), the C₁-C₆ alkyl group, the C₁-C₄ alkyl group, the alkyl moiety of the C₁-C₄ alkoxy group and the alkyl moiety of (R^{6d})₂N— may be substituted with a halogen atom, a hydroxyl group, an acetoxy group, a carbamoyl group, a cyano group, G, a 4-R^{6d}-piperazine-1-yl group (where R^{6d} may be identical or different), (R^{6d})₂N— (where R^{6d} may be identical or different), R^{6d}O— (where R^{6d} may be identical or different), a C₁-C₄ alkyl group, a C₁-C₄ alkoxy group, (R^{6d})₂NC(═O)— (where R^{6d} may be identical or different), R^{7d}C(═O)— (where R^{7d} may be identical or different), R^{5d}ONH— (where R^{5d} may be identical or different), R^{5d}NH— (where R^{5d} may be identical or different), R^{5d}NHC(═O)— (where R^{5d} may be identical or different), (R^{5d})₂NC(═O)-(where R^{5d} may be identical or different) or R^{5d}V (where R^{5d} may be identical or different). Furthermore, these substituents may be substituted with a halogen atom, a C₁-C₄ alkyl group, a C₁-C₄ alkoxy group, (R^{6d})₂N— (where R^{6d} may be identical or different), (R^{6d})₂NC(═O)— (where R^{6d} may be identical or different), R^{7d}C(═O) (where R^{7d} may be identical or different), R^{5d}ONH— (where R^{5d} may be identical or different), R^{5d}NH— (where R^{5d} may be identical or different), R^{5d}NHC(═O)— (where R^{5d} may be identical or different), (R^{5d})₂NC(═O)— (where R^{5d} may be identical or different), G or R^{5d}V (where R^{5d} may be identical or different). Two or more heteroatoms, however, cannot bind to the same carbon atom. Examples of heteroatoms include nitrogen, oxygen and sulfur atoms.

In General Formula (VII), three or less “R^{9d}” units may constitute R^{1d}.

In General Formula (VII), the benzamide group, the benzenesulfonylamino group, the phenyl group, the phenoxy group, the anilino group or the phenylsulfanyl group above in R^{1d} may have one or two halogen atoms, C₁-C₄ alkyl groups, cyano groups, methanesulfonyl groups or C₁-C₄ alkoxy groups as substituents, The alkyl group and the alkyl moiety of the alkoxy group or the alkylamino group may be liner or when they comprise three or more carbons, they may be a branched or cyclic 3-8-membered ring, preferably a 5-8-membered ring.

In General Formula (VII),

R^{2d} is selected from a hydrogen atom and an optionally substituted C₁-C₆ alkyl group;

t is 1 or 2,

each R^{3d} is independently selected from a hydrogen atom, an optionally substituted C₁-C₆ alkyl group, an optionally substituted amino group, a halogen atom, a hydroxyl group and an optionally substituted hydroxyl group;

R^{4d} independently represents a hydrogen atom, an azido group or a R^{11d}-ethynyl group, wherein R^{11d} is a hydrogen atom or an optionally substituted C₁-C₆ alkyl group, while the substituent is selected from a hydrogen atom, an amino group, a hydroxyl group, R^{5d}O— (where R^{5d} may be identical or different), R^{5d}NH— (where R^{5d} may be identical or different) and (R^{5d})₂N— (where R^{5d} may be identical or different).

In General Formula (VII), a “halogen atom” is preferably a fluorine atom, a chlorine atom, a bromine atom or an iodine atom.

In General Formula (VII), “C₁-C₄ alkyl group” is synonymous with the “C₁-C₄ alkyl group” described above.

In General Formula (VII), “C₁-C₄ alkoxy group” is synonymous with the “C₁-C₄ alkoxy group” described above.

In General Formula (VII), “C₂-C₄ alkenyl group” refers to a linear or branched alkenyl group having a double bond and a carbon number of 2-4, specific examples being an ethenyl group (a vinyl group), a 1-propenyl group, a 2-propenyl group (an allyl group), a 1-butenyl group, a 2-butenyl group and a 3-butenyl group.

In General Formula (VII), “(C₂-C₄)alkanoyl amino group” refers to, for example, a methylcarbonylamino group, an ethylcarbonylamino group, a n-propylcarbonylamino group and an isopropylcarbonylamino group.

In General Formula (VII), “(C₂-C₄)alkanoyloxy group” refers to, for example, a methylcarbonyloxy group, an ethylcarbonyloxy group, a n-propylcarbonyloxy group and an isopropylcarbonyloxy group.

Preferably, in General Formula (VII), s, t, R^{1d}, R^{3d} and R^{4d} are as defined above and R^{2d} is a hydrogen atom.

In General Formula (VII), more preferable compounds are
6,7-(dimethoxyquinazoline-4-yl)-(3-ethynylphenyl)-amine,
6,7-(dimethoxynazoline-4-yl)-[3-(3′-hydroxypropine-1′-yl)phenyl]-amine,
([3-(2′-aminomethyl)-ethynyl]phenyl)-(6,7-dimethoxyquinazoline-4-yl)-amine,
(3-ethynylphenyl)-(6-nitroquinazoline-4-yl)-amine,
(6,7-dimethoxyquinazoline-4-yl)-(4-ethynylphenyl)-amine,
(6,7-dimethoxyquinazoline-4-yl)-(3-ethynyl-2-methylphenyl)-amine,
(6-aminoquinazoline-4-yl)-(3-ethynylphenyl)-amine,
(3-ethynylphenyl)-(6-methanesulfonylaminoquinazoline-4-yl)-amine,
(3-ethynylphenyl)-(6,7-methylenedioxyquinazoline-4-yl)-amine,
(6,7-dimethoxyquinazoline-4-yl)-(3-ethynyl-6-methylphenyl)-amine,
(3-ethynylphenyl)-(7-nitroquinazoline-4-yl)-amine,
(3-ethynylphenyl)-[6-(4′-toluenesulfonylamino)quinazoline-4-yl]-amine,
(3-ethynylphenyl)-{6-[2′-phthalimide-ethane-1′-yl-sulfonylamino]quinazoline-4-yl}-amine,
(3-ethynylphenyl)-(6-guanidinoquinazoline-4-yl)-amine,
(7-aminoquinazoline-4-yl)-(3-ethynylphenyl)-amine,
(3-ethynylphenyl)-(7-methoxyquinazoline-4-yl)-amine,
(6-carbomethoxyquinazoline-4-yl)-(3-ethynylphenyl)-amine,
(7-carbomethoxyquinazoline-4-yl)-(3-ethynylphenyl)-amine,
[6,7-bis(2-methoxyethoxy)quinazoline-4-yl]-(3-ethynylphenyl)amine,
(3-azidophenyl)-(6,7-dimethoxyquinazoline-4-yl)-amine,
(4-azidophenyl)-(6,7-dimethoxyquinazoline-4-yl)-amine,
(3-azido-5-chlorophenyl)-(6,7-dimethoxyquinazoline-4-yl)-amine,
(3-ethynylphenyl)-(6-methanesulfonylquinazoline-4-yl)-amine,
(6-ethanesulfanyl-quinazoline-4-yl)-(3-ethynylphenyl)-amine,
(6,7-dimethoxy-quinazoline-4-yl)-(3-ethynyl-4-fluoro-phenyl)-amine,
(6,7-dimethoxy-quinazoline-4-yl)-[3-(propyl-1-yl-phenyl)]-amine,
[6,7-bis(2-methoxy-ethoxy)-quinazoline-4-yl]-(5-ethynyl-2-methyl-phenyl)-amine,
[6,7-bis(2-methoxy-ethoxy)-quinazoline-4-yl]-(3-ethynyl-4-fluoro-phenyl)-amine,
[6,7-bis(2-chloro-ethoxy)-quinazoline-4-yl]-(3-ethynyl-phenyl)-amine,
[6-(2-chloro-ethoxy)-7-(2-methoxy-ethoxy)-quinazoline-4-yl]-(3-ethynyl-phenyl)-amine,
[6,7-bis(2-acetoxy-ethoxy)-quinazoline-4-yl]-(3-ethynyl-phenyl)-amine,
2-[4-(3-ethynyl-phenylamino)-7-(2-hydroxy-ethoxy)-quinazoline-6-yloxy]-ethanol,
[6-(2-acetoxy-ethoxy)-7-(2-methoxy-ethoxy)-quinazoline-4-yl]-(3-ethynyl-phenyl)-amine,
[7-(2-chloro-ethoxy)-6-(2-methoxy-ethoxy)quinazoline-4-yl]-(3-ethynyl-phenyl)-amine,
[7-(2-acetoxy-ethoxy)-6-(2-methoxy-ethoxy)quinazoline-4-yl]-(3-ethynyl-phenyl)-amine,
2-[4-(3-ethynyl-phenylamino)-6-(2-hydroxy-ethoxy)-quinazoline-7-yloxy]-ethanol,
2-[4-(3-ethynyl-phenylamino)-7-(2-methoxy-ethoxy)-quinazoline-6-yloxy]-ethanol,
2-[4-(3-ethynyl-phenylamino)-6-(2-methoxy-ethoxy)-quinazoline-7-yloxy]-ethanol,
[6-(2-acetoxy-ethoxy)-7-(2-methoxy-ethoxy)-quinazoline-4-yl]-(3-ethynyl-phenyl)-amine,
(3-ethynyl-phenyl)-{6-(2-methoxy-ethoxy)-7-[2-(4-methyl-piperazine-1-yl)-ethoxy]-quinazoline-4-yl}-amine,
(3-ethynyl-phenyl)-[7-(2-methoxy-ethoxy)-6-(2-morpholine-4-yl)-ethoxy-quinazoline-4-yl]-amine,
(6,7-diethoxyquinazoline-4-yl)-(3-ethynylphenyl)-amine,
(6,7-dibutoxyquinazoline-4-yl)-(3-ethynylphenyl)-amine,
(6,7-diisopropoxyquinazoline-4-yl)-(3-ethynylphenyl)-amine,
(6,7-diethoxyquinazoline-4-yl)-(3-ethynyl-2-methylphenyl)-amine,
[6,7-bis(2-methoxy-ethoxy)-quinazoline-4-yl]-(3-ethynyl-2-methyl-phenyl)-amine,
(3-ethynylphenyl)-[6-(2-hydroxy-ethoxy)-7-(2-methoxy-ethoxy)-quinazoline-4-yl]amine,
[6,7-bis(2-hydroxy-ethoxy)-quinazoline-4-yl]-(3-ethynyl-2-methyl-phenyl)-amine,
2-[4-(3-ethynyl-phenylamino)-6-(2-methoxy-ethoxy)quinazoline-7-yloxy]-ethanol and 4-(3-ethynylphenylamino)-6,7-bis(2-methoxyethoxy)-quinazoline.

A compound represented by General Formula (VII) can be produced according to a known method, for example, by those described in International Publication No. 96/30347 (pamphlet) (WO96/30347), Japanese Patent No. 3088018 (JP3088018) and Japanese Patent No. 3420549 (JP3420549).

In General Formula (VII), a particularly preferable compound is erlotinib.

Erlotinib refers to 4-(3-ethynylphenylamino)-6,7-bis(2-methoxyethoxy)-quinazoline, whose structural formula is represented by the following Formula (XIV).

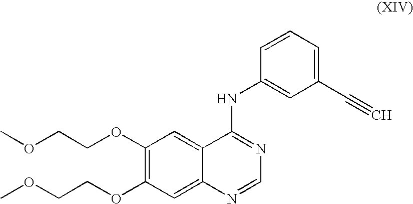

Erlotinib can be produced according to a known method, for example, by those described in International Publication No. 96/30347 (pamphlet) (WO96/30347), Japanese Patent No. 3088018 (JP3088018) and Japanese Patent No. 3420549 (JP3420549).

Erlotinib can also be obtained by purchasing Tarceva® from Genentech.

An example of EGFR kinase inhibitor includes lapatinib. Lapatinib refers to N-[3-chloro-4-[(3-fluorobenzyl)oxy]phenyl]-6-[5-[[[2-(methylsulfonyl)ethyl]amino]methyl]furan-2-yl]quinazoline-4-amine, whose structural formula is represented by the following Formula (XV).

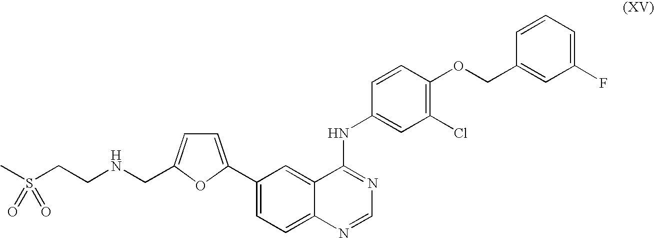

Lapatinib can be produced according to a known method, for example, by a method described in International Publication No. 99/35146 (pamphlet) (WO99/35146).

In addition, an example of lapatinib preferably includes lapatinib ditosylate. Lapatinib ditosylate refers to N-[3-chloro-4-[(3-fluorobenzyl)oxy]phenyl]-6-[5-[[[2-(methylsulfonyl)ethyl]amino]methyl]furan-2-yl]quinazoline-4-amine bis(4-methylbenzenesulfonate)monohydrate, whose structural formula is represented by the following Formula (XVI).

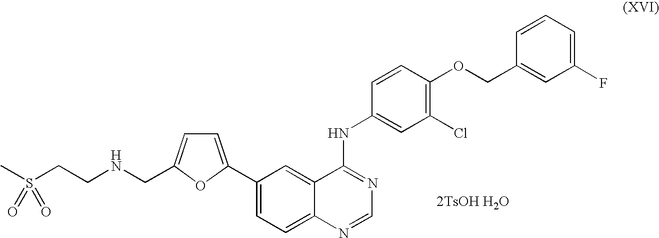

Lapatinib ditosylate can be produced according to a known method.

Furthermore, an example of EGFR kinase inhibitor includes canertinib. Canertinib refers to N-[4-[N-(3-chloro-4-fluorophenyl)amino]-7-[3-(4-morpholinyl)propoxy]quinazoline-6-yl]acrylamide (Clinical Cancer Research., 10:691-700, 2004), whose structural formula is represented by the following Formula (XVII).

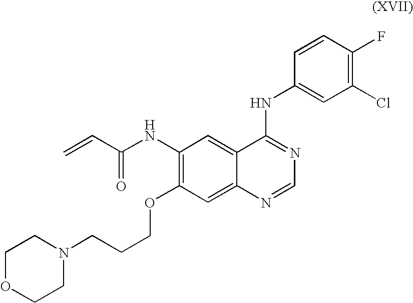

Canertinib can be produced according to a known method, for example, by those described in International Publication No. 2000/31048 (pamphlet) (WO2000/31048).

An example of preferable canertinib includes canertinib dihydrochloride. Canertinib dihydrochloride refers to N-[4-[N-(3-chloro-4-fluorophenyl)amino]-7-[3-(4-morpholinyl)propoxy]quinazoline-6-yl]acrylamide dihydrochloride, whose structural formula is represented by the following Formula (XVIII).

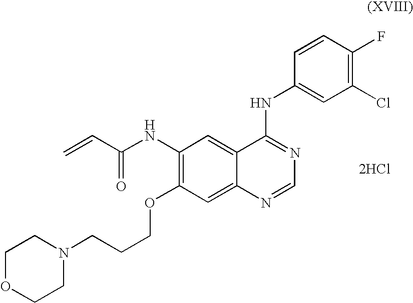

Canertinib dihydrochloride can be produced according to a known method.

Another example of EGFR kinase inhibitor is pelitinib. Pelitinib refers to (2E)-N-[4-[(3-chloro-4-fluorophenyl)amino]-3-cyano-7-ethoxy-6-quinolinyl]-4-(dimethylamino)-2-buteneamide (Methods Find Exp Clin Pharmacol., 27:49-77, 2005.), whose structural formula is represented by the following Formula (XIX).

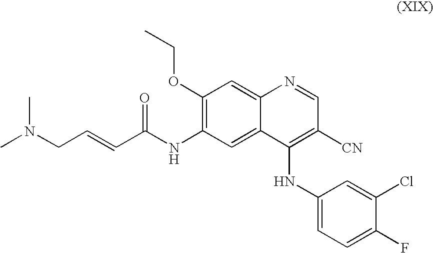

Pelitinib can be produced according to a known method, for example, by those described in International Publication No. 2003/50090 (pamphlet) (WO2003/50090).

Another example of EGFR kinase inhibitor is AEE-788. AEE-788 refers to [6-[4-[(4-ethylpiperazine-1-yl)methyl]phenyl]-7H-pyrrolo[2,3-d]pyrimidine-4-yl]-((R)-1-phenylethyl)amine (Cancer Research., 64, 4931-4941, 2004., Cancer Research., 64, 7977-7984, 2004), whose structural formula is represented by the following Formula (XX).

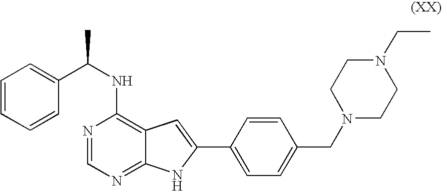

AEE-788 can be produced according to a known method, for example, by a method described in International Publication No. 2005/75460 (pamphlet) (WO2005/75460).

Another example of EGFR kinase inhibitor is HKI-272. HKI-272 refers to (E)-N-{4-[3-chloro-4-(2-pyridinylmethoxy)anilino]-3-cyano-7-ethoxy-6-quinolinyl}-4-(dimethylamino)-2-buteneamide (Cancer Research., 64, 3958-3965, 2004., Journal of Medicinal Chemistry., 48, 1107-1131, 2005), whose structural formula is represented by the following Formula (XXI).

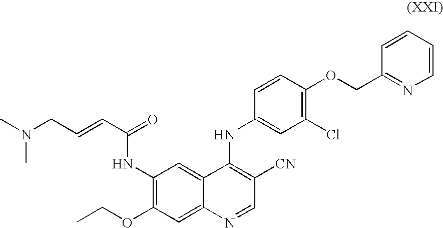

HKI-272 can be produced according to a known method, for example, by a method described in Journal of Medicinal Chemistry., 48, 1107-1131, 2005.

(2) Anti-EGFR Antibody

According to the present invention, an anti-EGFR antibody is preferably a neutralizing antibody that recognizes and binds to EGFR to inhibit EGF activity, preferably, cell-proliferative activity. According to the present invention, the degree of neutralization of EGF activity (cell-proliferative activity) by the anti-EGFR antibody is not particularly limited and any anti-EGFR antibody can be used as long as it can recognize and bind to EGFR and inhibit EGF activity. According to the present invention, the anti-EGFR antibody may be either a polyclonal antibody or a monoclonal antibody. The isotype of this antibody is not particularly limited and may be, for example, IgG (IgG₁, IgG₂, IgG₃, IgG₄), IgM, IgA (IgA₁, IgA₂), IgD or IgE.

The polyclonal antibody and the monoclonal antibody can be produced by a method well known to those skilled in the art (Antibodies: A Laboratory Manual, E. Harlow and D. Lane, ed., Cold Spring Harbor Laboratory (Cold Spring Harbor, N.Y., 1988)).

A polyclonal antibody can be obtained, for example, as follows: blood is drawn from a mammal such as mouse, a rabbit and a rat which has been administered with an antigen, and an antibody is separated and purified from the blood. Immunosensitization methods are known to those skilled in the art and can be carried out, for example, by single or multiple administrations of the antigen. Although the antigen (including part of or entire EGFR) may be dissolved upon use in an appropriate buffer, for example, an appropriate buffer containing a generally-used adjuvant such as complete Freund's adjuvant or aluminum hydroxide, an adjuvant may not be used depending on the route and conditions of administration.

One to two months after the last immunosensitization, blood is drawn from the mammal and subjected to separation and purification by a conventional method such as centrifugation, precipitation using ammonium sulfate or polyethyleneglycol and various chromatographies, thereby obtaining a polyclonal antibody as a polyclonal antiserum.

An example of a method for producing a monoclonal antibody includes a hybridoma method. First, similar to the production of the polyclonal antibody, a mammal is immunosensitized. After an appropriate period of days following the immunization, blood is partially drawn to determine the antibody titer preferably by a known method such as ELISA method.

Then, a spleen is excised from the immunized animal to obtain B cells. Subsequently, the B cells were fused with myeloma cell according to a conventional method to produce an antibody-producing hybridoma. The myeloma cell used is not limited to a particular kind and a known one can be used. The method for fusing the cells may be selected from methods known in the art such as Sendai virus method, polyethyleneglycol method and protoplast method. The obtained hybridoma is cultured according to a conventional method in a HAT medium (a medium containing hypoxanthine, aminopterin and thymidine) for an appropriate period of time for hybridoma selection. Then, after screening the antibody-producing hybridoma of interest, the hybridoma is cloned.

The screening method may be a known antibody detection method such as ELISA method and radioimmunoassay method, while the cloning method may be a method known in the art such as limiting dilution method and FACS method. The obtained hybridoma is cultured in an appropriate culture solution or administered to an organism compatible with the hybridoma, for example, intraperitoneally administered to a mouse. From the resulting culture solution or ascitic fluid, the intended monoclonal antibody can be isolated and purified by, for example, salt out, ion-exchange chromatography, gel filtration or affinity chromatography.

In addition, the fragment and the single-stranded antibody of the V region of the antibody can also be used in the present invention. The fragment of the antibody refers to a part of the polyclonal antibody or the monoclonal antibody described above, specific examples being F(ab′)₂, Fab′, Fab, Fv (variable fragment of antibody), sFv, dsFv (disulphide stabilized Fv) and dAb (single domain antibody). Moreover, an antibody used with the invention may be a chimeric antibody, a humanized antibody or a human antibody. The modified antibody can be produced according to a known method. A human antibody, for example, can be made in a similar manner as a usual monoclonal antibody by using a mammal having a human immune system.

A chimeric antibody is an antibody made from a variable (V) region of an antibody derived from a mammal other than human and a constant (C) region of a human antibody. A chimeric antibody can be obtained, for example, by linking DNA encoding a V region of an antibody derived from a mammal other than human with DNA encoding a C region of a human antibody, and integrating the resultant into an expression vector which is introduced into a host for production (European Patent Publication No. 125023 (specification) and International Publication No. 92/19759 (pamphlet)).

A humanized antibody is an antibody obtained by introducing at least one complementarity determining region (CDR) of an antibody derived from a mammal other than human into a human-antibody-derived CDR, and thus it contains a complementarity determining region derived from a mammal other than human and a framework region and C region of a human antibody. The gene of the humanized antibody can be produced, for example, by a general gene recombination technique (see, e.g., European Patent Publication No. 125023 (specification) and International Publication No. 92/19759 (pamphlet)).

According to the present invention, an anti-EGFR antibody is preferably cetuximab.

Cetuximab can be obtained according to methods described in Japanese Laid-Open Patent Publication No. 2002-114710 (JP2004-114710) and Japanese Laid-Open Patent Publication No. 2-291295 (JP2-291295).

Moreover, cetuximab can be obtained by purchasing Erbitux® from Merck and Bristol-Myers Squibb.

Another example of the anti-EGFR antibody is nimotuzumab. Nimotuzumab can be obtained according to methods described in European Patent No. 203126 (specification) (EP203126) and U.S. Pat. No. 5,891,996 (specification) (U.S. Pat. No. 5,891,996).

Another example of the anti-EGFR antibody is panitumumab (Clinical Colon Cancer. 2005; 5(1):21-3.). Panitumumab refers to an antibody registered as CAS 339177-26-3.

Another example of the anti-EGFR antibody is matuzumab (Curr Opin Mol Ther. 2004; 6(1):96-103.). Matuzumab refers to an antibody registered as CAS 339186-68-4.

Other examples of the anti-EGFR antibody are IMC-11F8 (Am. Assoc. Cancer Research, A5353, 2005) and MDX-447 (ASCO 18: 433, 1999). These antibodies can also be produced according to known methods described, for example, in documents shown in parentheses following the mentioned antibodies.

(3) Salts, Hydrates and Solvates

A substance having an EGF inhibitory activity may form a pharmacologically acceptable salt with acid or base. A substance having an EGF inhibitory activity of the invention also comprises these pharmacologically acceptable salts. Examples of salts formed with acids include inorganic acid salts such as hydrochloride salts, hydrobromide salts, sulfate salts and phosphate salts, and salts formed with organic acids such as formic acid, acetic acid, lactic acid, succinic acid, fumaric acid, maleic acid, citric acid, tartaric acid, benzoic acid, methanesulfonic acid, benzenesulfonic acid, p-toluenesulfonic acid and trifluoroacetic acid. Examples of salts formed with bases include alkali metal salts such as sodium salt and potassium salt, alkaline earth metal salts such as calcium salt and magnesium salt, and salts formed with organic bases such as trimethylamine, triethylamine, pyridine, picoline, dicyclohexylamine and N,N′-dibenzylethylenediamine, arginine, lysine (organic amine salts), and ammonium salts.

A substance having an EGF inhibitory activity may be an anhydride or may form a solvate such as a hydrate. The solvate may be either a hydrate or a nonhydrate, preferably a hydrate. The solvent may use water, alcohol (e.g., methanol, ethanol or n-propanol), dimethylformamide or the like.

If solvates and/or enantiomers of these substances having an EGF inhibitory activity exist, they are also comprised in the substance having an EGF inhibitory activity of the invention. The substance having an EGF inhibitory activity of the invention also comprises substances having an EGF inhibitory activity that undergo metabolism in vivo such as oxidation, reduction, hydrolysis and conjugation. Moreover, the substance having an EGF inhibitory activity of the invention also comprises compounds that generate substances having an EGF inhibitory activity by undergoing metabolism in vivo such as oxidation, reduction and hydrolysis.

3. Pharmaceutical Composition, Kit and Method for Treating Cancer

The present invention relates to a pharmaceutical composition, a kit and a method for treating cancer, characterized by comprising a sulfonamide compound in combination with a substance having an EGF inhibitory activity.

According to the present invention, a sulfonamide compound is as described in “1. Sulfonamide compound”. For example, the sulfonamide compound is at least one compound selected from: (A) a compound represented by General Formula (I), preferably E7070 or E7820; (B) a compound represented by General Formula (II), preferably LY186641 or LY295501; (C) a compound represented by General Formula (III), preferably LY-ASAP; (D) LY573636 (Formula (IV)) and (E) CQS (Formula (V)). More preferably, the sulfonamide compound is at least one compound selected from LY295501 and LY573636 and more preferably sodium salt of LY573636.

According to the present invention, a sulfonamide compound is preferably E7070 or E7820.

According to the present invention, a substance having an EGF inhibitory activity is as described in “2. Substance having EGF inhibitory activity”. For example, the substance having an EGF inhibitory activity is at least one substance selected from: (A) an EGF receptor kinase inhibitor, preferably gefitinib, erlotinib, lapatinib, canertinib, pelitinib, AEE-788 or HKI-272; and (B) an anti-EGFR antibody, preferably cetuximab, panitumumab, matuzumab, nimotuzumab, IMC-11F8 or MDX-447. More preferably, the substance having an EGF inhibitory activity is at least one substance selected from gefitinib, erlotinib and cetuximab.

According to the present invention, the sulfonamide compound and the substance having an EGF inhibitory activity also comprise pharmacologically acceptable salts thereof, or solvates such as hydrates thereof.

According to the present invention, the sulfonamide compound and the substance having an EGF inhibitory activity may be used in any combination.

The pharmaceutical composition of the invention comprises a sulfonamide compound in combination with a substance having an EGF inhibitory activity. The pharmaceutical composition of the invention is useful for treating cancer.

According to the present invention, the term “in combination with” refers to a combination of compounds for combinational use, and includes both modes in which separate compounds are administered in combination and as a mixture.

The pharmaceutical composition of the invention is also provided in another embodiment of a pharmaceutical composition comprising a sulfonamide compound, which is administered to a patient in combination with a substance having an EGF inhibitory activity. The sulfonamide compound and the substance having an EGF inhibitory activity may be administered either simultaneously or separately. The term “simultaneous” refers to administrations at the same timing in a single administration schedule. In this case, it is not necessary to use completely the same hour and minute for administration. The term “separately” refers to administrations at different timings in a single administration schedule.

The kit of the invention comprises a set of a formulation comprising a sulfonamide compound and a formulation comprising a substance having an EGF inhibitory activity. The formulations comprised in the kit of the invention are not limited to a particular form as long as they comprise a sulfonamide compound or a substance having an EGF inhibitory activity. The kit of the invention is useful for treating cancer.

In the kit of the invention, the formulation comprising a sulfonamide compound and the formulation comprising a substance having an EGF inhibitory activity may be mixed together or separately accommodated in a single package. They may be administered simultaneously or one may be administered preceding the other.

The pharmaceutical composition and/or the kit and the method for treating cancer of the invention may be further combined with one or more additional anti-cancer drugs. The additional anti-cancer drugs are not particularly limited as long as they are formulations having an anti-tumor activity. Examples of the additional anti-cancer drugs include irinotecan hydrochloride (CPT-11), oxaliplatin, 5-fluorouracil (5-FU), docetaxel (Taxotere®), gemcitabine hydrochloride (Gemzar®), calcium folinate (Leucovorin) and bevacizumab (Avastin®). Particularly preferable additional anti-cancer drugs are irinotecan hydrochloride, oxaliplatin, 5-fluorouracil, calcium folinate or bevacizumab when the type of cancer to be treated by the drug is colon cancer, gemcitabine hydrochloride or bevacizumab for pancreas cancer, bevacizumab for renal cancer, and docetaxel for lung cancer.

More examples of particularly preferable combinations of the compounds according to the invention are shown in Tables 1, 2, 3 and 4 for the cases of treating colon cancer, pancreas cancer, renal cancer and lung cancer by the therapeutic drug, respectively.

TABLE 1

| | Combined Compounds | | | | | |
|---|---|---|---|---|---|---|
| 1 | E7070 | Gefitinib | 5-FU | LV | Oxaliplatin | |
| 2 | E7820 | Gefitinib | 5-FU | LV | Oxaliplatin | |
| 3 | E7070 | Erlotinib | 5-FU | LV | Oxaliplatin | |
| 4 | E7820 | Erlotinib | 5-FU | LV | Oxaliplatin | |
| 5 | E7070 | Cetuximab | 5-FU | LV | Oxaliplatin | |
| 6 | E7820 | Cetuximab | 5-FU | LV | Oxaliplatin | |
| 7 | E7070 | Gefitinib | 5-FU | LV | Oxaliplatin | Bevacizumab |
| 8 | E7820 | Gefitinib | 5-FU | LV | Oxaliplatin | Bevacizumab |
| 9 | E7070 | Erlotinib | 5-FU | LV | Oxaliplatin | Bevacizumab |
| 10 | E7820 | Erlotinib | 5-FU | LV | Oxaliplatin | Bevacizumab |
| 11 | E7070 | Cetuximab | 5-FU | LV | Oxaliplatin | Bevacizumab |
| 12 | E7820 | Cetuximab | 5-FU | LV | Oxaliplatin | Bevacizumab |
| 13 | E7070 | Gefitinib | 5-FU | LV | CPT-11 | |
| 14 | E7820 | Gefitinib | 5-FU | LV | CPT-11 | |
| 15 | E7070 | Erlotinib | 5-FU | LV | CPT-11 | |
| 16 | E7820 | Erlotinib | 5-FU | LV | CPT-11 | |
| 17 | E7070 | Cetuximab | 5-FU | LV | CPT-11 | |
| 18 | E7820 | Cetuximab | 5-FU | LV | CPT-11 | |
| 19 | E7070 | Gefitinib | 5-FU | LV | CPT-11 | Bevacizumab |
| 20 | E7820 | Gefitinib | 5-FU | LV | CPT-11 | Bevacizumab |
| 21 | E7070 | Erlotinib | 5-FU | LV | CPT-11 | Bevacizumab |
| 22 | E7820 | Erlotinib | 5-FU | LV | CPT-11 | Bevacizumab |
| 23 | E7070 | Cetuximab | 5-FU | LV | CPT-11 | Bevacizumab |
| 24 | E7820 | Cetuximab | 5-FU | LV | CPT-11 | Bevacizumab |
| 25 | E7070 | Gefitinib | Bevacizumab | | | |
| 26 | E7820 | Gefitinib | Bevacizumab | | | |
| 27 | E7070 | Erlotinib | Bevacizumab | | | |
| 28 | E7820 | Erlotinib | Bevacizumab | | | |
| 29 | E7070 | Cetuximab | Bevacizumab | | | |
| 30 | E7820 | Cetuximab | Bevacizumab | | | |

Table 1 shows preferable combinations of the invention where the type of cancer to be treated by the therapeutic drug for cancer is colon cancer. In the table, LV represents calcium folinate.

TABLE 2

Combined Compounds

| | | | | |
|---|---|---|---|---|
| 1 | E7070 | Gefitinib | Gemcitabine | |
| 2 | E7820 | Gefitinib | Gemcitabine | |
| 3 | E7070 | Erlotinib | Gemcitabine | |
| 4 | E7820 | Erlotinib | Gemcitabine | |
| 5 | E7070 | Cetuximab | Gemcitabine | |
| 6 | E7820 | Cetuximab | Gemcitabine | |
| 7 | E7070 | Gefitinib | Gemcitabine | Bevacizumab |
| 8 | E7820 | Gefitinib | Gemcitabine | Bevacizumab |
| 9 | E7070 | Erlotinib | Gemcitabine | Bevacizumab |
| 10 | E7820 | Erlotinib | Gemcitabine | Bevacizumab |
| 11 | E7070 | Cetuximab | Gemcitabine | Bevacizumab |
| 12 | E7820 | Cetuximab | Gemcitabine | Bevacizumab |

Table 2 shows preferable combinations of the invention where the type of cancer to be treated by the therapeutic drug for cancer is pancreas cancer. In the table, Gemcitabine represents gemcitabine hydrochloride.

TABLE 3

Combined Compounds

| | | | |
|---|---|---|---|
| 1 | E7070 | Gefitinib | Bevacizumab |
| 2 | E7820 | Gefitinib | Bevacizumab |
| 3 | E7070 | Erlotinib | Bevacizumab |
| 4 | E7820 | Erlotinib | Bevacizumab |
| 5 | E7070 | Cetuximab | Bevacizumab |
| 6 | E7820 | Cetuximab | Bevacizumab |

Table 3 shows preferable combinations of the invention where the type of cancer to be treated by the therapeutic drug for cancer is renal cancer.

TABLE 4

Combined Compounds

| | | | |
|---|---|---|---|
| 1 | E7070 | Gefitinib | Docetaxel |
| 2 | E7820 | Gefitinib | Docetaxel |
| 3 | E7070 | Erlotinib | Docetaxel |
| 4 | E7820 | Erlotinib | Docetaxel |
| 5 | E7070 | Cetuximab | Docetaxel |
| 6 | E7820 | Cetuximab | Docetaxel |

Table 4 shows preferable combinations of the invention where the type of cancer to be treated by the therapeutic drug for cancer is lung cancer.

The pharmaceutical composition and/or the kit of the invention can be used as a therapeutic drug for cancer.

Treatments according to the present invention comprise symptomatic relief of the disease, progression delay of symptoms of the disease, elimination of the symptoms of the disease, improvement of prognosis of the disease, and prevention of recurrence of the disease.

A therapeutic drug for cancer according to the invention comprises those that contain an anti-tumor agent, a drug for improving prognosis of cancer, a drug for preventing cancer recurrence, an antimetastatic drug or the like.

The effect of cancer treatment can be confirmed by observation of X-ray pictures, CT or the like, histopathologic diagnosis by biopsy, or tumor marker value.

The pharmaceutical composition and/or the kit of the invention can be administered to mammals (e.g., human, rat, rabbit, sheep, pig, cattle, cat, dog and monkey).

Examples of the types of cancers targeted by the therapeutic drug for cancer include but not limited to at least one selected from the group consisting of brain tumor, cervical cancer, esophageal cancer, tongue cancer, lung cancer, breast cancer, pancreas cancer, gastric cancer, small intestinal and duodenal cancer, colon cancer (colon cancer and rectal cancer), bladder cancer, renal cancer, liver cancer, prostate cancer, uterine cancer, ovarian cancer, thyroid grand cancer, gallbladder cancer, pharyngeal cancer, sarcoma (e.g., osteosarcoma, chondrosarcoma, Kaposi's sarcoma, myosarcoma, angiosarcoma, fibrosarcoma, etc.), leukemia (e.g., chronic myelocytic leukemia (CML), acute myelocytic leukemia (AML), chronic lymphocytic leukemia (CLL), acute lymphocytic leukemia (ALL), lymphoma, multiple myeloma (MM), etc.) and melanoma. Preferably, the type of cancer targeted by the therapeutic drug for cancer is at least one selected from the group consisting of colon cancer, pancreas cancer, renal cancer and lung cancer, more preferably lung cancer, and particularly preferably non-small-cell lung cancer.

The pharmaceutical composition and/or the kit of the invention may be administered orally or parenterally.

When the pharmaceutical composition and/or kit of the invention is used, the given dose of the sulfonamide compound differs depending on the degree of the symptom, age, sex, weight and sensitivity difference of the patient, administration mode, administration period, administration interval, and nature, prescription and type of the pharmaceutical formulation and the type of the active ingredient. Usually, but without limitation, the dose of the sulfonamide compound is 10-6000 mg/day, preferably 50-4000 mg/day, more preferably 50-2000 mg/day for an adult (weight 60 Kg), which may be administered once to three times a day.

When using the pharmaceutical composition and/or the kit of the invention, the given dose of the substance having an EGF inhibitory activity is usually, but not particularly limited to, 10-6000 mg/day, preferably 50-4000 mg/day, more preferably 50-2000 mg/day for an adult, which may be administered once to three times a day.

When using the pharmaceutical composition and/or the kit of the invention, the given dose of the EGFR kinase inhibitor is usually, but not particularly limited to, 10-6000 mg/day, preferably 50-4000 mg/day, more preferably 50-2000 mg/day for an adult, which may be administered once to three times a day.

When using the pharmaceutical composition and/or the kit of the invention, the given dose of the anti-EGFR antibody is usually, but not particularly limited to, 1-6000 mg/day, preferably 10-2000 mg/day, more preferably 10-1000 mg/day for an adult, which may be administered once to three times a day.

The amount of the sulfonamide compound used is not particularly limited, and differs depending on the individual combination with a substance having an EGF inhibitory activity, preferably an EGFR kinase inhibitor or an anti-EGFR antibody. For example, the amount of the sulfonamide compound is about 0.01-100 times (weight ratio), more preferably about 0.1-10 times (weight ratio) of the amount of the substance having an EGF inhibitory activity, preferably an EGFR kinase inhibitor or an anti-EGFR antibody.

The pharmaceutical composition of the invention may be made into various dosage forms, for example, into solid oral formulations or parenteral formulations such as injection, suppository, ointment and skin patch.

Furthermore, the sulfonamide compound and the substance having an EGF inhibitory activity contained in the kit of the invention may individually be made into various dosage forms, for example, into solid oral formulations or parenteral formulations such as injection, suppository, ointment and skin patch.

In order to prepare a solid oral formulation, an excipient, and if necessary, a binder, disintegrant, lubricant, colorant, a flavoring agent or the like may be added to a principal agent, and then made into a tablet, a coated tablet, granule, subtle granule, powder, a capsule or the like according to a conventional method. In addition, a non-solid oral formulation such as a syrup agent can also be prepared appropriately.

For example, lactose, cornstarch, sucrose, glucose, sorbit, crystalline cellulose, silicon dioxide or the like may be used as the excipient; for example, polyvinyl alcohol, ethyl cellulose, methyl cellulose, gum arabic, hydroxypropyl cellulose, hydroxypropylmethyl cellulose or the like may be used as the binder; for example, magnesium stearate, talc, silica or the like may be used as the lubricant; those that are allowed to be added to pharmaceutical preparations may be used as the colorant; and for example, cocoa powder, menthol, aromatic acid, peppermint oil, camphor, cinnamon powder or the like may be used as the flavoring agent. Of course, if necessary, these tablets and granule may be coated appropriately with sugar coating, gelatin coating or else.

When an injection is to be prepared, if necessary, the principal agent may be added with a pH adjuster, a buffer, a suspending agent, a solubilizing aid, a stabilizer, an isotonizing agent, a preservative or the like, and may be made into an intravenously, subcutaneously or intramuscularly injection or an intravenous drip injection according to a conventional method. In this case, if necessary, it is also prepared a lyophilized form by a conventional technique.

Examples of the suspending agent may include methyl cellulose, Polysorbate 80, hydroxyethyl cellulose, gum arabic, powdered tragacanth, sodium carboxy methyl cellulose and polyoxyethylene sorbitan monolaurate.

Examples of the solubilizing aid may include polyoxyethylene hydrogenated castor oil, Polysorbate 80, nicotine acid amide, polyoxyethylene sorbitan monolaurate, macrogol, and ethyl ester of castor oil fatty acid.

Examples of the stabilizer may include sodium sulfite and sodium metasulfite; examples of the preservative may include methyl parahydroxybenzoate, ethyl parahydroxybenzoate, sorbic acid, phenol, cresol and chlorocresol.

Besides the sulfonamide compound and the substance having an EGF inhibitory activity, the pharmaceutical composition and/or the kit of the invention can also comprise a packaging container, an instruction, a package insert or the like. The packaging container, the instruction, the package insert or the like may include description of combinations for combinational use of the compound, and usage and dosage in the case of administering separate substances in combination or in the case of administering them in the form of a mixture. The usage and dosage may be described referring to the related description above.

The kit of the invention may also comprise: (a) at least one selected from the group consisting of a packaging container, an instruction and a package insert describing combinational use of the sulfonamide compound and the substance having an EGF inhibitory activity; and (b) a pharmaceutical composition comprising the sulfonamide compound. The kit is useful for treating cancer. The pharmaceutical composition comprising the sulfonamide compound is useful for treating cancer. The packaging container, the instruction, the package insert or the like may include the description of combinations for combinational use of the sulfonamide compound and the substance having an EGF inhibitory activity, and usage and dosage for combinational use in the case of administering separate substances in combination or in the case of administering them in the form of a mixture. The usage and dosage may be described referring to the related description above.

The present invention also comprises use of a sulfonamide compound for producing a pharmaceutical composition in combination with a substance having an EGF inhibitory activity. According to the use of the invention, the pharmaceutical composition is useful for treating cancer.

The present invention also comprises a method for treating cancer comprising simultaneously or separately administering a sulfonamide compound and a substance having an EGF inhibitory activity to a patient. According to the method of the invention for treating cancer, the route and the method for administering the sulfonamide compound and the substance having an EGF inhibitory activity are not particularly limited but reference may be made to the description of the pharmaceutical composition of the invention above.

Hereinafter, the present invention will be described by way of specific examples, although the present invention is not limited thereto.

### EXAMPLE 1

### Combinational Use of E7820 and Gefitinib on Proliferation of Human Non-Small-Cell Lung Cancer Cell Line (PC9) In Vitro

Human non-small-cell lung cancer cell line PC9 (obtained from Immuno-Biological laboratories Co., Ltd.) was suspended in RPMI1640 (containing 10% FBS) to 1×10⁴ cells/ml, and 100 μl each of this solution was added to each well of a 96 well plate for cultivation in a 5% carbon dioxide incubator at 37° C. Six hours after the start of the cultivation, the medium was removed. Then, a solution containing E7820, a solution containing gefitinib (Iressa® purchased from AstraZeneca) and a solution containing both compounds, i.e., E7820 and gefitinib, were each diluted in a culture solution (RPMI1640 (containing 10% FBS)). These diluted solutions were added to the cells above for further cultivation.

Three days later, 10 μl of cell counting kit-8 solution (Cell Counting Kit-8, Wako Pure Chemical Industries) was added, cultured for 6 hours at 37° C., and absorbance at 450 nm was determined with a plate reader (Corona Electric Co., Ltd.).

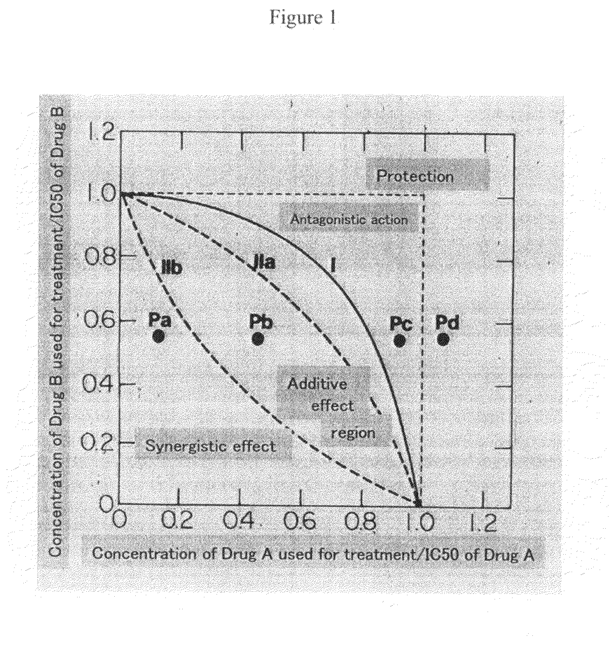

The effect of combinational use was assessed by Isobologram method (FIG. 1, Steel G G et al: Int J Radiat Oncol Biol Phys 5: 85-91, 1979., Kano Y, et al: Int J Cancer 50: 604-610, 1992.). According to this method, three types of curves (mode 1, mode IIa and mode IIb) were calculated from the curve of growing cell counts with respect to the concentration of each compound, showing theoretical concentrations for inhibiting 50% cell growth by combinational use. Therefore, when a plot of 50% inhibitory concentration (IC50) in the well where the compounds are used in combination is within the region surrounded by these three lines (additive region) (FIG. 1, “Pb”), it is considered to have an additive effect. When a plot of 50% inhibitory concentration (IC50) in a well where the compounds are used in combination is within a region innermost to the mode curves (FIG. 1, “Pa”), it is considered to have a synergistic effect. When a plot of 50% inhibitory concentration (IC50) in a well where the compounds are used in combination is within a region outside the mode curves (FIG. 1, “Pc”), it is considered to have an antagonistic effect. When a plot of 50% inhibitory concentration (IC50) in a well where the compounds are used in combination exceeds the IC50 of each compound, it is considered to have a protection effect (FIG. 1, “Pd”).

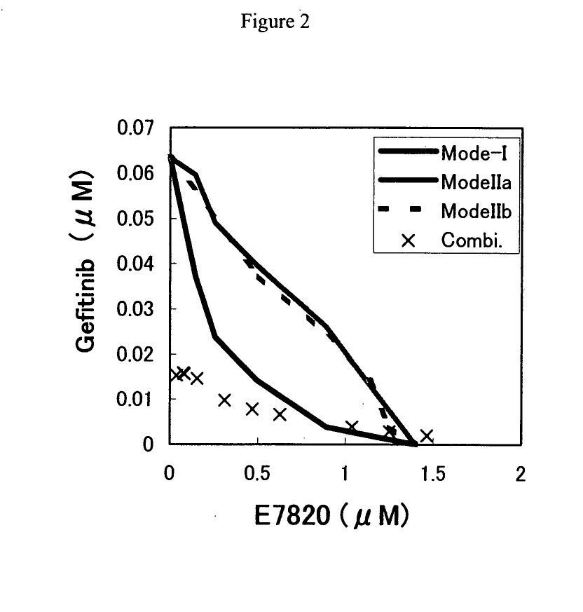

As a result, E7820 was found to have a synergistic effect upon combinational use with gefitinib (FIG. 2, “Combi.”).

### EXAMPLE 2

### Combinational Use of E7070 and Gefitinib on In Vitro Proliferarion of Human Non-Small-Cell Lung Cancer Cell Line (PC9)

Human non-small-cell lung cancer cell line PC9 was suspended in RPMI1640 (containing 10% FBS) to 1×10⁴ cells/ml, and 100 μl each of this solution was added to each well of a 96 well plate for cultivation in a 5% carbon dioxide incubator at 37° C. Twenty-four hours after the start of the cultivation, the medium was removed. A solution containing E7070, a solution containing gefitinib (purchased from AstraZeneca) and a solution containing both compounds, i.e., E7070 and gefitinib, were each diluted in a culture solution (RPMI1640 (containing 10% FBS)). Then, these diluted solutions were added to the cells above for further cultivation.

Three days later, the cells were washed with 100 μl PBS/well, and immobilized with 10% trichloroacetic acid. Then, the cells were stained by SRB technique to determine absorbance at 550 nm with a plate reader.

The effect of combinational use was assessed by Isobologram method.

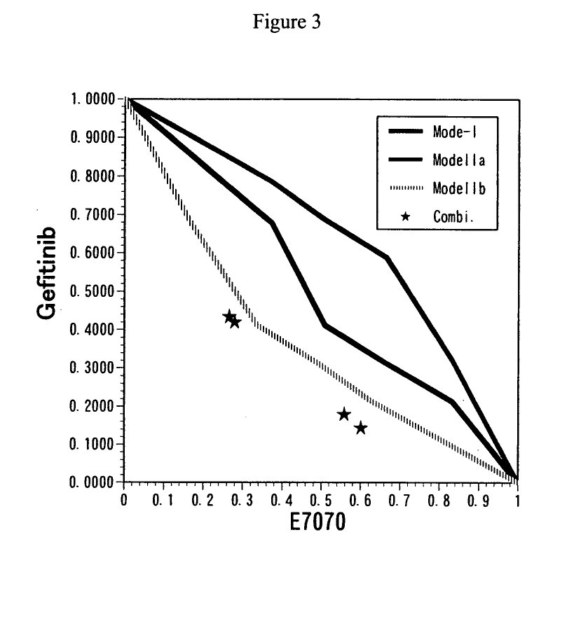

As a result, E7070 was found to have a synergistic effect upon combinational use with gefitinib (FIG. 3, “Combi.”)).

### EXAMPLE 3

### Combinational Use of E7070 and Erlotinib on In Vitro Proliferarion of Human Non-Small-Cell Lung Cancer Cell Line (PC9)

Human non-small-cell lung cancer cell line PC9 was suspended in RPMI1640 (containing 10% FBS) to 1×10⁴ cells/ml, and 100 μl each of this solution was added to each well of a 96 well plate for cultivation in a 5% carbon dioxide incubator at 37° C. Twenty-four hours after the start of the cultivation, the medium was removed. A solution containing E7070, a solution containing erlotinib (Tarceva® purchased from Genentech) and a solution containing both compounds, i.e., E7070 and erlotinib, were each diluted in a culture solution (RPMI1640 (containing 10% FBS)). Then, these diluted solutions were added to the cells above for further cultivation.

Three days later, the cells were washed with 100 μl PBS/well, and immobilized with 10% trichloroacetic acid. Then, the cells were stained by SRB technique to determine absorbance at 550 nm with a plate reader.

The effect of combinational use was assessed by Isobologram method.

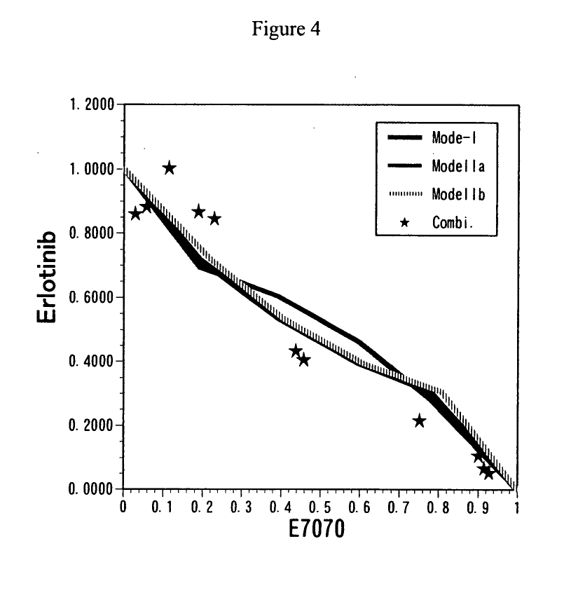

As a result, E7070 was found to have a synergistic effect upon combinational use with erlotinib (FIG. 4, “Combi.”)).

### EXAMPLE 4

### Combinational Use of E7820 and Gefitinib in Subcutaneous Transplant Model (In Vivo) of Human Non-Small-Cell Lung Cancer Cell Line (PC9)

Human non-small-cell lung cancer cell line PC9 (obtained from Immuno-Biological laboratories Co., Ltd.) was cultured in RPMI1640 (containing 10% FBS) in a 5% carbon dioxide incubator at 37° C. to about 80% confluence, and the cells were collected with trypsin-EDTA. Using a phosphate buffer, 5×10⁷ cells/mL suspension was prepared, and 0.1 mL each of the resulting cell suspension was subcutaneously transplanted to a nude mouse at the side of its body. Eight days after the transplantation, E7820 and gefitinib were orally administered alone or in combination for 50 mg/kg twice a day for 2 weeks and for 75 mg/kg once a day for 2 weeks, respectively. The major and minor axes of tumors were measured with Digimatic caliper (Mitsutoyo), and tumor volumes and relative tumor volumes were calculated according to the following formulae.

Tumor Volume TV=Major axis of tumor(mm)×(Minor axis of tumor)²(mm²)/2

Relative Tumor Volume RTV=Tumor volume on measurement day/Tumor volume on the first administration day

If statistically significant interaction was observed in the combinational use group by two-way ANOVA, a synergistic effect was considered to exist between E7820 and gefitinib.

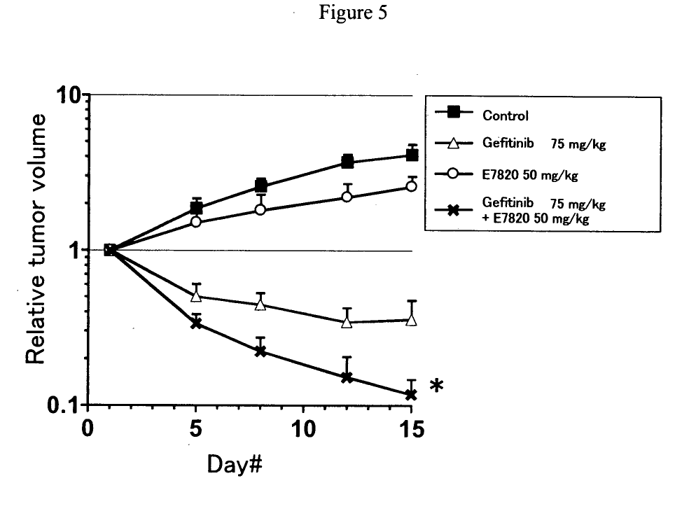

As a result, E7820 was found to produce a synergistic effect when used in combination with gefitinib, and their combinational use showed a superior anti-tumor effect as compared with the effect obtained with E7820 or gefitinib alone (Table 5 and FIG. 5). In addition, combinational use of E7820 and gefitinib also showed a remarkable anti-tumor effect that cannot be seen with gefitinib alone (Table 5 and FIG. 5).

TABLE 5

| Administered | Relative tumor volume on Day 15 | Two-way |
|---|---|---|
| compound | Average ± standard deviation | ANOVA |
| Control (untreated) | 4.12 ± 0.68 | |
| E7820 50 mg/kg | 2.57 ± 0.41 | |
| Gefitinib 75 mg/kg | 0.36 ± 0.12 | |
| E7820 50 mg/kg + | 0.12 ± 0.03 | p < 0.01 |
| gefitinib 75 mg/kg | | Synergistic |
| | | effect |

Table 5 shows anti-tumor effects obtained by the use of E7820 alone, the use of gefitinib alone and the combinational use of E7820 and gefitinib in subcutaneous transplant models of human non-small-cell lung cancer cell line (PC9). The first day of administration was considered Day 1.

From the obtained results, the combination of E7820 and gefitinib provides a pharmaceutical composition and a kit that show a remarkable anti-tumor activity, and a method for treating cancer, and thus the pharmaceutical composition, the kit and the method of the invention can be used for treating cancer.

### EXAMPLE 5

### Combinational Use of E7820 and Gefitinib in Subcutaneous Transplant Model (In Vivo) of Human Non-Small-Cell Lung Cancer Cell Line (A549)

Human non-small-cell lung cancer cell line A549 (purchased from Dainippon Pharmaceutical) was cultured in RPMI1640 (containing 10% FBS) in a 5% carbon dioxide incubator at 37° C. to about 80% confluence, and the cells were collected with trypsin-EDTA. Using a phosphate buffer containing 50% matrigel, 5×10⁷ cells/mL suspension was prepared, and 0.1 mL each of the resulting cell suspension was subcutaneously transplanted to a nude mouse at the side of its body. Ten days after the transplantation, E7820 and gefitinib were orally administered alone or in combination for 50 mg/kg twice a day for 3 weeks and for 75 mg/kg once a day for 3 weeks, respectively. The major and minor axes of tumors were measured with Digimatic caliper (Mitsutoyo), and tumor volumes and relative tumor volumes were calculated according to the following formulae.

Tumor Volume TV=Major axis of tumor(mm)×(minor axis of tumor)²(mm²)/2

Relative Tumor Volume RTV=Tumor volume on measurement day/Tumor volume on the first administration day

If statistically significant interaction was observed in the combinational use group by two-way ANOVA, a synergistic effect is considered to exist between E7820 and gefitinib.

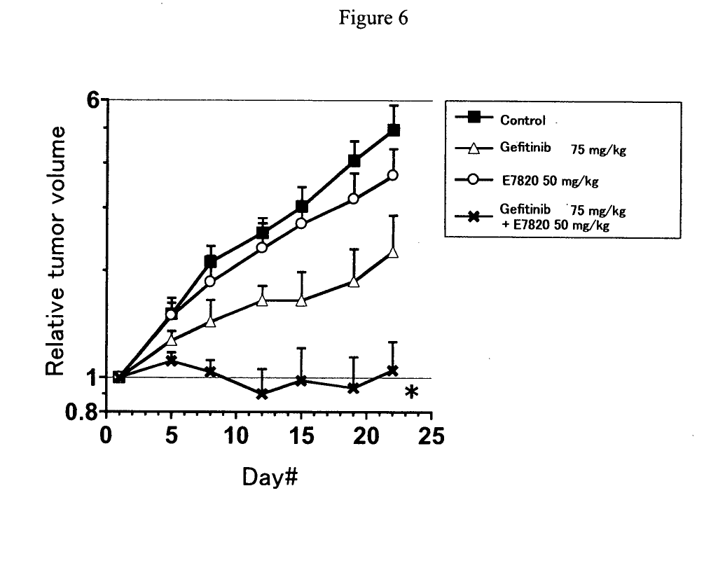

As a result, E7820 was found to produce a synergistic effect when used in combination with gefitinib, and their combinational use showed a superior anti-tumor effect as compared with the effect obtained with E7820 or gefitinib alone (Table 6 and FIG. 6). In addition, combinational use of E7820 and gefitinib also showed a remarkable anti-tumor effect that cannot be seen with gefitinib alone (Table 6 and FIG. 6).

TABLE 6

| | Relative | |
|---|---|---|
| Administered | tumor volume on Day 22 | Two-way |
| compound | Average ± standard deviation | ANOVA |
| Control (untreated) | 4.95 ± 0.86 | |
| E7820 50 mg/kg | 3.69 ± 0.68 | |
| Gefitinib 75 mg/kg | 2.26 ± 0.59 | |
| E7820 50 mg/kg + | 1.05 ± 0.21 | p < 0.01 |
| gefitinib 75 mg/kg | | Synergistic effect |

Table 6 shows anti-tumor effects obtained by the use of E7820 alone, the use of gefitinib alone and the combinational use of E7820 and gefitinib in subcutaneous transplant models of human non-small-cell lung cancer cell line (A549). The first day of administration was considered Day 1.

From the obtained results, the combination of E7820 and gefitinib provides a pharmaceutical composition and a kit that show a remarkable anti-tumor activity, and a method for treating cancer, and thus the pharmaceutical composition, the kit and the method of the invention can be used for treating cancer.

### EXAMPLE 6

### Combinational Use of E7820 and Erlotinib in Subcutaneous Transplant Model (In Vivo) of Human Non-Small-Cell Lung Cancer Cell Line (A549)

Human non-small-cell lung cancer cell line A549 (purchased from Dainippon Pharmaceutical) was cultured in RPMI1640 (containing 10% FBS) in a 5% carbon dioxide incubator at 37° C. to about 80% confluence, and the cells were collected with trypsin-EDTA. Using a phosphate buffer containing 50% matrigel, 5×10⁷ cells/mL suspension was prepared, and 0.1 mL each of the resulting cell suspension was subcutaneously transplanted to a nude mouse at the side of its body. Seventeen days after the transplantation, E7820 and erlotinib were orally administered alone or in combination for 50 mg/kg twice a day for 2 weeks and for 100 mg/kg once a day for 2 weeks, respectively. The major and minor axes of tumors were measured with Digimatic caliper (Mitsutoyo), and tumor volumes and relative tumor volumes were calculated according to the following formulae.

Tumor Volume TV=Major axis of tumor(mm)×(Minor axis of tumor)²(mm²)/2

Relative Tumor Volume RTV=Tumor volume on measurement day/Tumor volume on the first administration day

If statistically significant interaction was observed in the combinational use group by two-way ANOVA, a synergistic effect was considered to exist between E7820 and erlotinib.

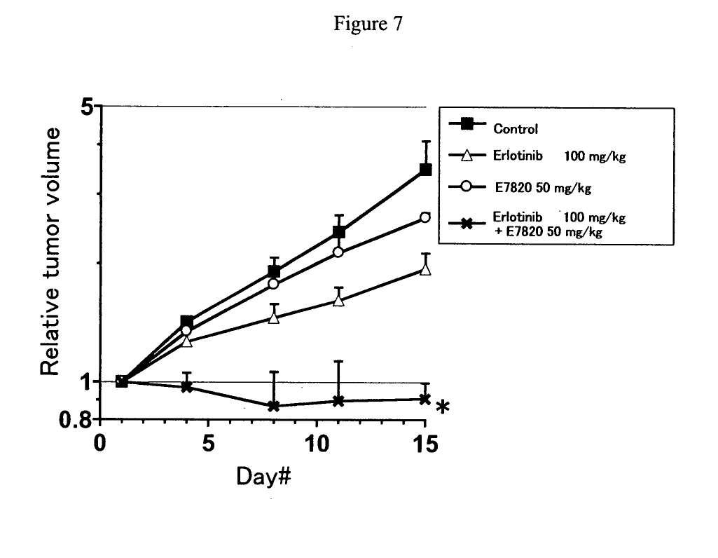

As a result, E7820 was found to produce a synergistic effect when used in combination with erlotinib and their combinational use showed a superior anti-tumor effect as compared with the effect obtained with E7820 or erlotinib alone (Table 7 and FIG. 7). In addition, combinational use of E7820 and erlotinib also showed a remarkable anti-tumor effect that cannot be seen with erlotinib alone (Table 7 and FIG. 7).

TABLE 7

| | Relative | |
|---|---|---|
| Administered | tumor volume on Day 15 | Two-way |
| compound | Average ± standard deviation | ANOVA |
| Control (untreated) | 3.48 ± 0.61 | |
| E7820 50 mg/kg | 2.62 ± 0.07 | |
| Erlotinib 100 mg/kg | 1.94 ± 0.19 | |
| E7820 50 mg/kg + | 0.91 ± 0.09 | p < 0.01 |
| erlotinib 100 mg/kg | | Synergistic effect |

Table 7 shows anti-tumor effects obtained by the use of E7820 alone, the use of erlotinib alone and the combinational use of E7820 and erlotinib in subcutaneous transplant models of human non-small-cell lung cancer cell line (A549). The first day of administration was considered Day 1.

From the obtained results, the combination of E7820 and erlotinib provides a pharmaceutical composition and a kit that show a remarkable anti-tumor activity, and a method for treating cancer, and thus the pharmaceutical composition, the kit and the method of the invention can be used for treating cancer.

### EXAMPLE 7

### DNA Microarray Analysis

(1) Cell Culture, Compound Treatment and RNA Extraction

For the purpose of examining changes in the gene expression induced by the compounds by a DNA microarray analysis, human colon cancer-derived cell line HCT116 (American Type Culture Collection, Manassas, Va., U.S.A.) and human leukemia-derived cell line MOLT-4 (American Type Culture Collection, Manassas, Va., U.S.A.) were cultured in RPMI-1640 media supplemented with 10% fetal bovine serum, 100 units/ml penicillin and 100 μg/ml streptomycin. The following cultivation and compound treatment took place in an incubator set to 5% CO₂ and 37° C. The HCT116 cells and the MOLT-4 cells were seeded on 10 cm-diameter cell culture dishes at 2.0×10⁶ cells/dish, cultured for 24 hours and subjected to the following compound treatments.

For the HCT116 cells, 12 compounds, i.e., E7820 (0.8 μM), E7070 (0.8 μM), LY295501 (30 μM), CQS (8 μM), adriamycin (0.2 μM), daunomycin (0.2 μM), ICRF154 (80 μM), ICRF159 (80 μM), kenpaullone (10 μM), alsterpullone (10 μM), trichostatin A (0.1 μM) and rapamycin (80 μM) were assessed. On the other hand, for the MOLT-4 cells, E7070 (0.8 μM) was assessed. Herein, adriamycin and daunomycin are compounds known as DNA intercalative DNA topoisomerase II inhibitors, ICRF154 and ICRF159 are compounds known as catalytic DNA topoisomerase II inhibitors, kenpaullone and alsterpullone are compounds known as cyclin-dependent kinase (CDK) inhibitors, trichostatin A is a compound known as a histone deacetylase inhibitor and rapamycin is a compound known as an mTOR/FRAP inhibitor. The concentration of each compound used for the treatment was set to three to five-fold the 50% growth inhibitory concentration of each compound to the HCT116 cells (based on three days of antiproliferative activity using WST-8). The cells were collected 24 hours after the treatment at the concentration indicated in parentheses following each compound name above. Similarly, cells cultured for 24 hours without the addition of any compound were also collected.

Extraction of total RNA from the collected cells was performed using TRIZOL reagent (Invitrogen) according to the attached instruction.

(2) Analysis of Gene Expression Using DNA Microarray

The resulting RNA was dissolved in 100 μl of diethylpyrocarbonate (DEPC)-treated sterilized water, purified using an RNeasy column (QIAGEN), and double-stranded cDNA was synthesized using SuperScript Choice System (Invitrogen) and T7-d(T)₂₄ primers.

First, to 10 μg RNA, 5 μM T7-d(T)₂₄ primer, 1× First strand buffer, 10 mM DTT, 500 μM dNTP mix and 20 units/μl SuperScript II Reverse Transcriptase were added and reacted at 42° C. for an hour to synthesize single-stranded DNA. Subsequently, 1× Second strand buffer, 200 μM dNTP mix, 67 U/ml DNA ligase, 270 U/ml DNA polymerase I and 13 U/ml RNase H were added and reacted at 16° C. for two hours to synthesize double-stranded cDNA. Furthermore, 67 U/ml T4 DNA polymerase I was added, reacted at 16° C. for 5 minutes and then 10 μl of 0.5 M EDTA was added to terminate the reaction.

The obtained cDNA was purified with phenol/chloroform, and subjected to labeling reaction with biotinylated UTP and CTP using RNA Transcript Labeling Kit (Enzo Diagnostics) according to the attached instruction. The reaction product was purified using an RNeasy column, heated in 200 mM Tris acetic acid (pH8.1), 150 mM magnesium acetate and 50 mM potassium acetate at 94° C. for 35 minutes for fragmentation of the cRNA.

The fragmented cRNA was hybridized to GeneChip (Affymetrix) Human Focus array in 100 mM MES, 1 M sodium salt, 20 mM EDTA and 0.01% Tween 20 at 45° C. for 16 hours. After the hybridization, GeneChip was washed and stained according to protocol Midi_euk2 attached to the Affymetrix fluidics station. For staining, streptavidin-phycoerythrin and biotinylated anti-streptavidin goat antibody were used. The stained GeneChip was scanned using HP confocal microscope with argon ion laser (Hewlett Packard) to determine fluorescence intensity. Measurement took place at excitation and emission wavelengths of 488 nm and 570 nm, respectively.

All of the quantitative data analyses were carried out using GeneChip software (Affymetrix) and Gene Spring (Silicongenetics). GeneChip software was used for assessing changes in the gene expression induced by each compound, where gene expression was judged to have significantly “increased” or “decreased” when the quantified values in the two conditions, i.e., between the compound-treated group and the untreated group, were twice or more as different. Gene Spring was used for assessing the similarity of changes in gene expression induced by each compound, where hierarchical cluster analysis was conducted based on changes in the expressions of all genes on the Human Focus Array.

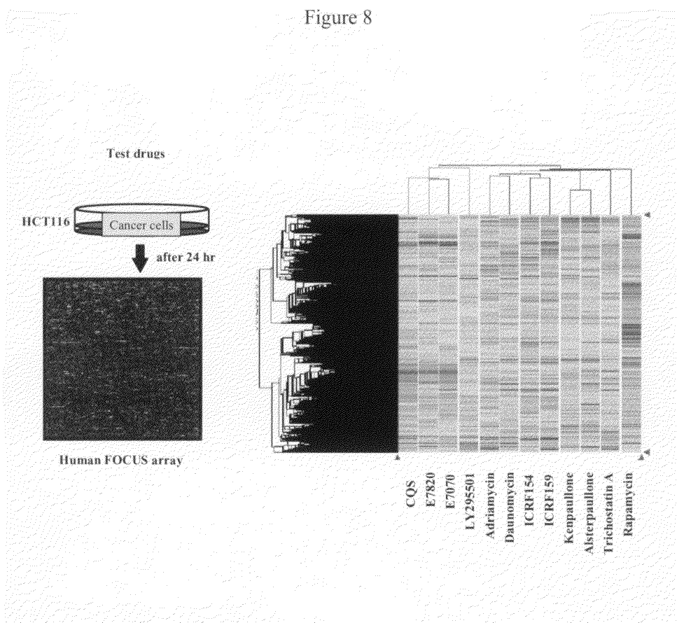

The results from the hierarchical cluster analysis for the HCT116 cells are shown in FIG. 8.

As a result of the analysis, adriamycin and daunomycin, ICRF154 and ICRF159, and Kenpaullone and alsterpullone, each pair having the same action mechanism, gave similar genetic alterations (FIG. 8). Thus, compounds having the same action mechanism were confirmed to give similar genetic alterations.

E7070, E7820, LY295501 and CQS gave similar genetic alterations (FIG. 8). Therefore, E7070, E7820, LY295501 and CQS were considered to have the same or similar action mechanisms according to this analysis, strongly suggesting that they give the same or similar genetic alterations and effects.

### EXAMPLE 8

### DNA Microarray Analysis

HCT116 cells were cultured in an RPMI-1640 medium supplemented with 10% fetal bovine serum, 100 units/ml penicillin and 100 μg/ml streptomycin. The following cultivation and compound treatment were carried out in an incubator at 5% CO₂ and 37° C. HCT116 cells were seeded in 10 cm-diameter cell culture dishes at 2.0×10⁶ cells/dish, cultured for 24 hours and subjected to the following compound treatment.

In this example, changes in the gene expression of HCT116 cells upon treatments with 12 compounds, i.e., E7820 (0.16 μM), E7070 (0.26 μM), LY186641 (59 μM), LY295501 (24 μM), LY-573636 (9.6 μM), CQS (4.0 μM), MST16 (100 μM), etoposide (3.6 μM), ethoxzolamide (410 μM), capsaicin (280 μM), trichostatin A (0.16 μM) and kenpaullone (7.1 μM) were examined.

MST16 is a compound known as a catalytic DNA topoisomerase II inhibitor, etoposide is a compound known as a DNA topoisomerase II inhibitor that induces formation of a cleavable complex, ethoxzolamide is a compound known as a carbonic anhydrase inhibitor, capsaicin is a compound known as a tumor-specific plasma membrane NADH oxidase inhibitor, trichostatin A is a compound known as a histone deacetylase inhibitor and kenpaullone is a compound known as a cyclin-dependent kinase (CDK) inhibitor.

The concentration of each compound used for the treatment was set to twice the 50% growth inhibitory concentration of each compound to the HCT116 cells (based on three days of antiproliferative activity using MTT). The cells were collected 24 hours after the treatment at the concentration indicated in parentheses following each compound name above. Similarly, cells cultured for 24 hours without the addition of any compound were also collected.

Total RNA extraction from the collected cells was performed using TRIZOL reagent (Invitrogen) according to the attached instruction.

Gene expression analysis using a DNA microarray was carried out in the same manner as “(2) Analysis of gene expression using DNA microarray” in Example 7.

This example was conducted for each sample in duplicate (for the convenience of the experiment, samples were given branch numbers like control-1, control-2, E7070-1, E7070-2 and so on for distinction). Then, GeneChip (Affymetrix) system (Human Focus array) was used for analyzing changes in the gene expression induced by each compound.

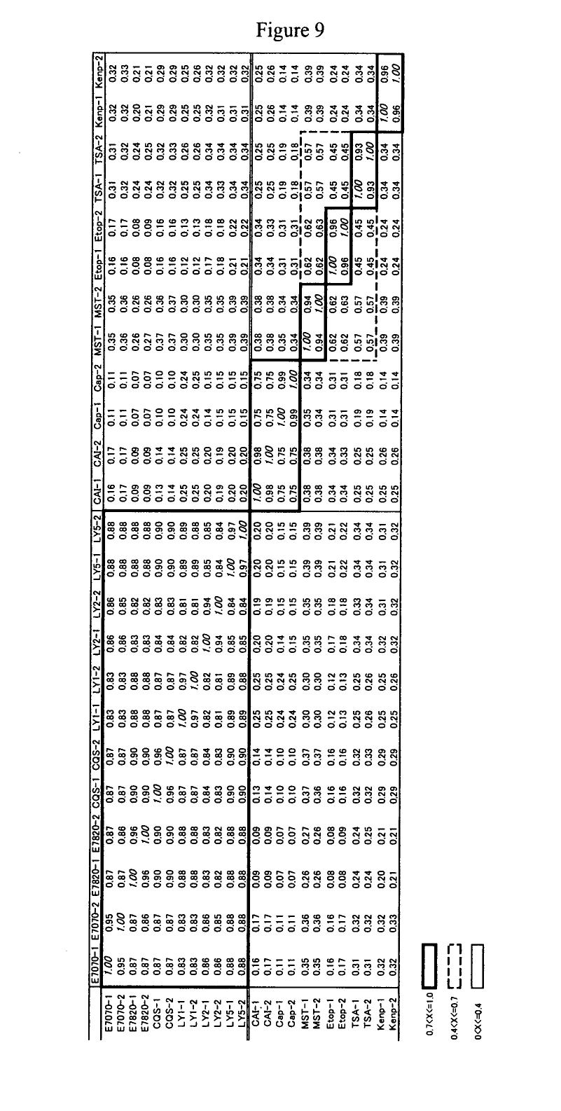

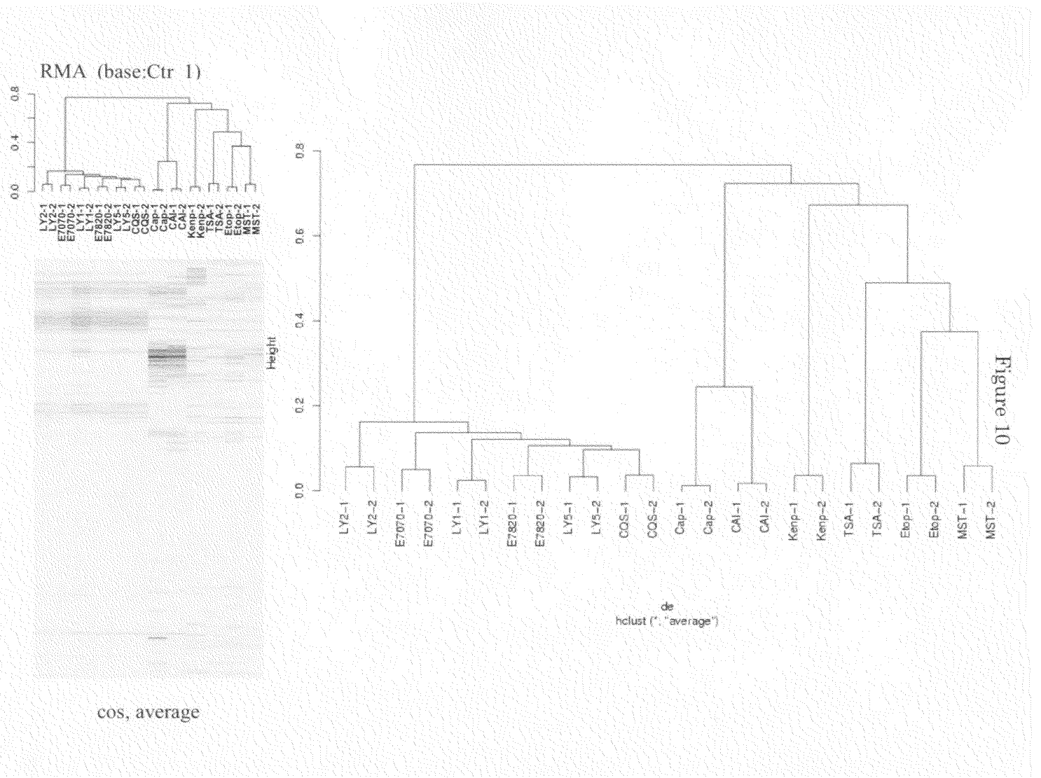

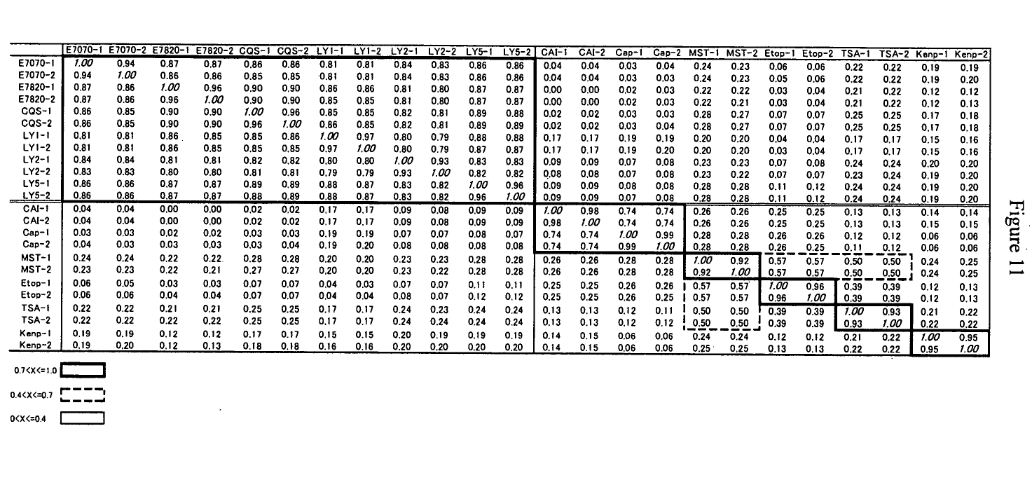

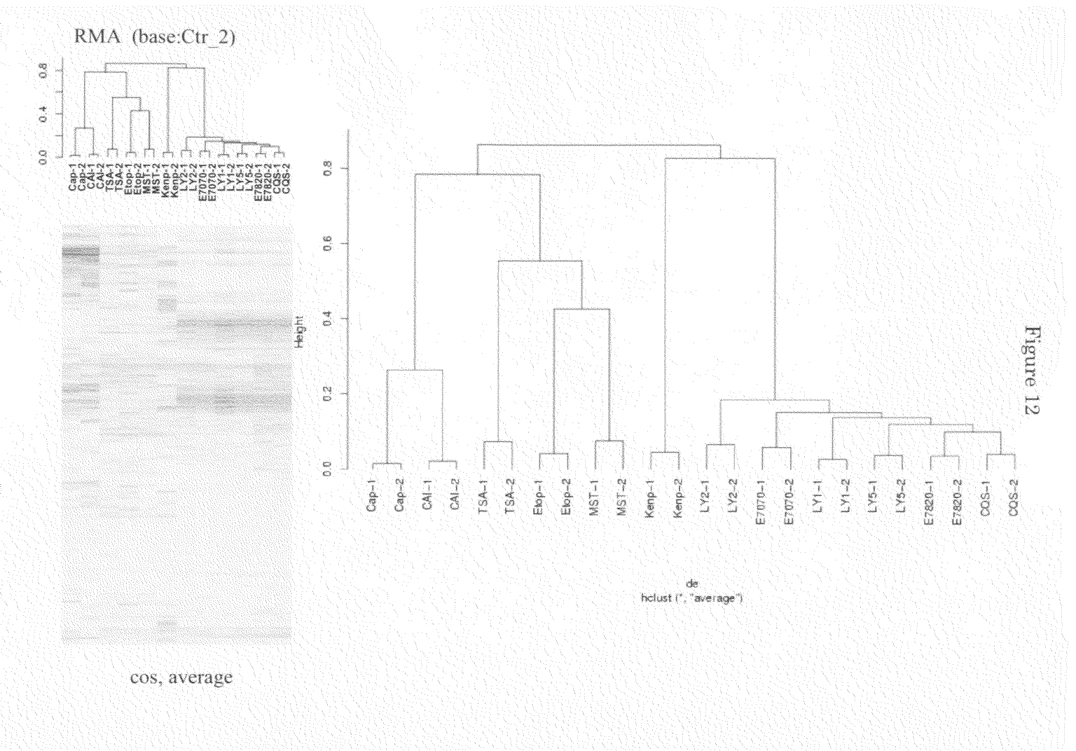

Twenty-six “.cel” files obtained in this example (13 samples (a control+12 compounds)×2) were subjected to RMA method (robust multi-array average method (Biostatistics (2003), 4, 249-264)) for normal distribution at probe level, and then the logarithm value of the signal intensity at gene level was calculated. Next, the logarithm value of the signal intensity of the untreated group (control-1) was subtracted from the logarithm value of the signal intensity of the compound-treated group for each gene to obtain the logarithm value of the signal ratio of the compound-treated group to control-1. Then, cosine correlation coefficients were calculated as correlation coefficients between the experiments (FIG. 9). Based on these correlation coefficients, hierarchical cluster analysis was performed according to UPGMA method (Unweighted Pair Group Method with Arithmetic mean method) (FIG. 10). Control-2 was also subjected to similar calculation (FIGS. 11 and 12). The softwares used were R 2.0.1 (http://www.r-project.org/) and affy package 1.5.8 (http://www.bioconductor.org).

In FIGS. 9-12, “LY1” represents LY186641, “LY2,” represents LY295501, “LY5” represents LY573636, “CAI” represents ethoxzolamide, “Cap” represents capsaicin, “MST” represents MST16, “Etop” represents etoposide, “TSA” represents trichostatin A, and “Kenp” represents kenpaullone. In FIGS. 10 and 12, “de hclust (*, “average”)” is a command upon statistical analysis, showing that clustering analysis is conducted by R using the average value of the duplicate experiment data.

As a result of the analysis, E7070, E7820, LY186641, LY295501, LY573636 and CQS showed very similar genetic alterations for the HCT116 cells, and were found to be different from the profiles of any of the other compounds (MST16, etoposide, ethoxzolamide, capsaicin, trichostatin A and kenpaullone) (FIGS. 9-12). Thus, by this analysis, E7070, E7820, LY186641, LY295501, LY573636 and CQS were considered to have the same or similar action mechanisms, strongly suggesting that they give the same or similar genetic alterations and effects.

### EXAMPLE 9

### Experiment on Cancer Cell Line Panels

Human cancer cell panels from 36 cell lines were used to examine correlation of antiproliferative activities among E7820, E7070, CQS, LY186641 and LY295501. The 36 types of cancer cell lines used were DLD-1, HCT15, HCT116, HT29, SW480, SW620 and WiDr (which are human colon cancer cell lines), A427, A549, LX-1, NCI-H460, NCI-H522, PC-9 and PC-10 (which are human lung cancer cell lines), GT3TKB, HGC27, MKN1, MKN7, MKN28 and MKN74 (which are human gastric cancer cell lines), AsPC-1, KP-1, KP-4, MiaPaCall, PANC-1 and SUIT-2 (which are human pancreas cancer cell lines), BSY-1, HBC5, MCF-7, MDA-MB-231, MDA-MB-435 and MDA-MB-468 (which are human breast cancer cell lines), and CCRF-CEM, HL60, K562 and MOLT-4 (which are human leukemia cell lines). All of the cells were cultured using RPMI-1640 media supplemented with 10% fetal bovine serum, 100 units/ml penicillin and 100 μg/ml streptomycin under the conditions of 5% CO₂ and 37° C. (Table 8).

TABLE 8
36 human cancer cell lines tested in 3-day MTT assays

| Colon | Stomach | Breast |
|---|---|---|
| DLD-1 (1250/well, 16.8 h) | GT3TKB (2000/well, 21.1 h) | BSY-1 (2000/well, 46.1 h) |
| HCT15 (1500/well, 14.5 h) | HGC27 (1500/well, 14.6 h) | HBC5 (2000/well, 31.8 h) |
| HCT116 (1250/well, 13.4 h) | MKN1 (4000/well, 35.9 h) | MCF-7 (3000/well, 29.5 h) |
| HT29 (2500/well, 19.8 h) | MKN7 (3000/well, 37.4 h) | MDA-MB231 (2000/well, 21.6 h) |
| SW480 (3000/well, 19.5 h) | MKN28 (2000/well, 22.7 h) | MDA-MB-435 (3000/well, 24.4 h) |
| SW620 (2500/well, 17.3 h) | MKN74 (4000/well, 24.8 h) | MDA-MB-468 (3000/well, 34.2 h) |
| WiDr (2000/well, 18.9 h) | | |

| Lung | Pancreas | Leukemia |
|---|---|---|
| A427 (2500/well, 32.4 h) | AsPC-1 (2500/well, 28.4 h) | CCRF-CEM (1500/well, 27.2 h) |
| A549 (1250/well, 18.9 h) | KP-1 (2000/well, 24.8 h) | HL60 (1500/well, 29.5 h) |
| LX-1 (2000/well, 17.2 h) | KP-4 (2000/well, 16.7 h) | K562 (1500/well, 20.6 h) |
| NCI-H460 (1000/well, 13.6 h) | MiaPaCaII (2500/well, 19.1 h) | MOLT-4 (1500/well, 22.3 h) |
| NCI-H522 (4000/well, 80.4 h) | PANC-1 (2500/well, 27.9 h) | |
| PC-9 (2000/well, 23.7 h) | SUIT-2 (2000/well, 15.6 h) | |
| PC-10 (2000/well, 24.0 h) | | |

### Cell Line (Initial Cell Number, Doubling Time)

Table 8 shows the types, seeded cell numbers and doubling times of the human cancer cell lines in the human cancer cell line panels.

The cells were seeded on a 96-well microplate (flat bottom) at the number indicated in Table 8 (50 μl/well). Twenty-four hours later, they were added with a 3-fold dilution series of each compound (50 μl/well). Seventy-two hours later, WST-8 (10 μl/well) was added and absorbance at 450 nm was determined. The 50% growth inhibitory concentrations to all of the 36 cancer cell lines were obtained by a least square method and their patterns were compared between the compounds. As the correlation index, Pearson's correlation coefficients were used (Paull, K. D. et al. Display and analysis of patterns of differential activity of drugs against human tumor cell lines: development of mean graph and COMPARE algorithm. J. Natl. Cancer Inst. 1989, 81, 1088-1092; Monks, A. et al. Feasibility of a high-flux anticancer drug screen using a diverse panel of cultured human tumor cell lines. J. Natl. Cancer Inst. 1991, 83, 757-766).

As a result, E7070, E7820, LY186641, LY295501 and CQS showed high correlation coefficients in antiproliferative activities against each cancer cell line (Table 9). Thus, by this analysis, E7070, E7820, LY186641, LY295501 and CQS were considered to have the same or similar action mechanisms, strongly suggesting that they give the same or similar genetic alterations and effects.

TABLE 9

| | E7070 | E7820 | CQS | LY186641 | LY295501 |
|---|---|---|---|---|---|
| E7070 | 1.00 | 0.98 | 0.97 | 0.93 | 0.80 |
| E7820 | 0.98 | 1.00 | 0.96 | 0.95 | 0.82 |
| CQS | 0.97 | 0.96 | 1.00 | 0.92 | 0.82 |
| LY186641 | 0.93 | 0.95 | 0.92 | 1.00 | 0.81 |
| LY295501 | 0.80 | 0.82 | 0.82 | 0.81 | 1.00 |

Table 9 shows correlation coefficients between the compounds (E7070, E7820, CQS, LY186641 and LY295501) on the human cancer cell line panels.

### EXAMPLE 10

### Cross-Resistance in E7070-Resistant Cell Line

An E7070-resistant cell line was used to assess the antiproliferative activities of E7820, LY186641, LY295501, LY-ASAP and CQS. HCT116-C9 was a substrain separated from human colon cancer-derived HCT116 (American Type Culture Collection, Manassas, Va., U.S.A.). This HCT116-C9 was cultured in the presence of E7070 while increasing the E7070 concentration by degrees, thereby obtaining E7070-resistant substrains HCT116-C9-C1 and HCT116-C₉-C₄ (Molecular Cancer Therapeutics, 2002, 1, 275-286).

Three cell lines, i.e., HCT116-C9, HCT116-C9-C1 and HCT116-C₉-C₄, were each seeded at 3000 cells/well onto a 96-well microplate (flat bottom) (50 μl/well). Twenty-four hours later, they were added with a 3-fold dilution series of each compound (50 μl/well). Seventy-two hours later, the antiproliferative activities were assessed by MTT method (Mossmann T., J. Immunol. Methods, 1983, 65, 55-63). The 50% growth inhibitory concentrations to the cancer cells were obtained by a least square method.

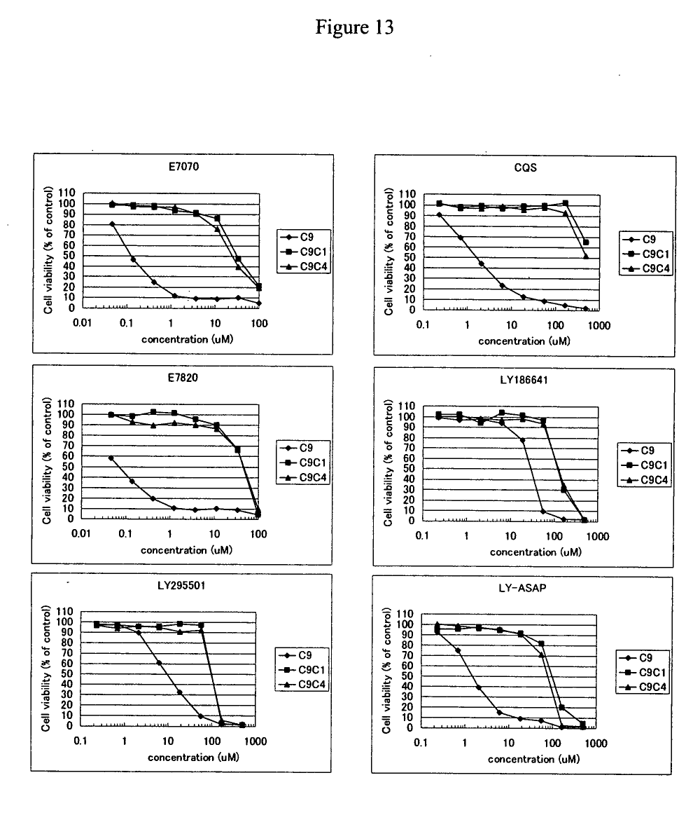

As a result, the antiproliferative activity, i.e., IC50, of E7070 to HCT116-C9 (C9) was 0.127 μM. On the other hand, activities to HCT116-C9-C1 (C9C1) and HCT116-C₉-C₄ (C9C4) were IC50=31.9 μM and 26.9 μM, respectively, confirming that the antiproliferative activities of E7070 to C9C1 and C9C4 were remarkably low (FIG. 13). The antiproliferative activities of E7820, CQS, LY186641, LY295501 and LY-ASAP to HCT116-C9 were IC50=0.080 μM, 1.73 μM, 33.6 μM, 10.9 μM and 1.63 μM, respectively while their activities to HCT116-C9-C1 were IC50=51.2 μM, 634 μM, 134 μM, 111 μM and 113 μM, respectively and their activities to HCT116-C₉-C₄ were IC50=52.8 μM, 517 μM, 138 μM, 110 μM and 90.3 M, respectively. Therefore, the antiproliferative activities of E7820, CQS, LY186641, LY295501 and LY-ASAP to C9C1 and C9C4 were far lower than those to C9 (FIG. 13). Thus, E7070, E7820, LY186641, LY295501, LY-ASAP and CQS were considered to have the same or similar action mechanisms, strongly suggesting that they give the same or similar genetic alterations and effects.

### EXAMPLE 11

### Cross-Resistance in E7070-Resistant Cell Line

In exactly the same manner as in Example 10, an E7070-resistant cell line was used to assess the antiproliferative activities of LY573636 as well as those of E7070.

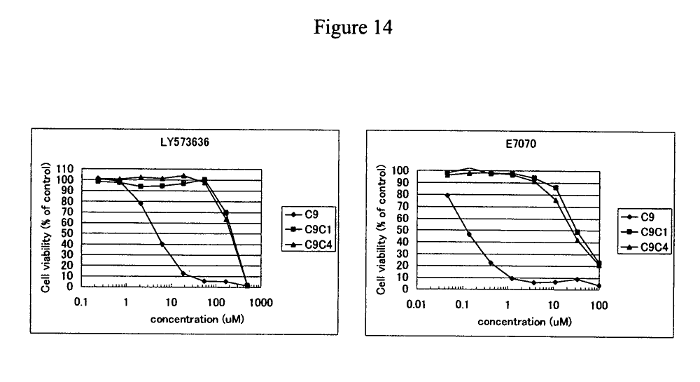

As a result, the antiproliferative activities of E7070 to HCT116-C9-C1 and HCT116-C₉-C₄ (IC50=32.7 μM and 28.0 μM, respectively) were again confirmed to be remarkably lower than the activity to HCT116-C9 (IC50=0.127 μM) (FIG. 14). The antiproliferative activities of LY573636 to HCT116-C9-C1 and HCT116-C₉-C₄ (IC50=264 μM and 240 μM, respectively) were also remarkably lower than the activity to HCT116-C9 (IC50=5.11 μM) (FIG. 14). Thus, LY573636 was considered to have the same or similar action mechanism as that of E7070, strongly suggesting that it gives the same or similar genetic alteration and effect.

These results (Examples 7-11) confirmed that E7070, E7820, LY186641, LY295501, LY-ASAP, LY573636, CQS or a combination thereof give the same or similar genetic alterations and thus the same or similar actions and effects.

Accordingly, similar to E7820 and E7070 (Examples 1-6, 12 and 13), a sulfonamide compound, preferably E7820, E7070, LY186641, LY295501, LY-ASAP, LY573636, CQS or a combination thereof was found to show a remarkable anti-tumor activity upon combinational use with a substance having an EGF inhibitory activity, preferably gefitinib, erlotinib or cetuximab.

### EXAMPLE 12

### Combinational Use of E7820 and Cetuximab in Subcutaneous Transplant Model (In Vivo) of Human Colon Cancer Cell Line (WiDr)

Human colon cancer cell line WiDr (obtained from Dainippon Pharmaceutical) was cultured in RPMI1640 (containing 10% FBS) in a 5% carbon dioxide incubator at 37° C. to about 80% confluence, and the cells were collected with trypsin-EDTA. Using a phosphate buffer, 5×10⁷ cells/mL suspension was prepared, and 0.1 mL each of the resulting cell suspension was subcutaneously transplanted to a nude mouse at the side of its body. Ten days after the transplantation, E7820 and cetuximab (Erbitux® purchased from Merck) were administered alone or in combination. E7820 was orally administered at 50 mg/kg twice a day for 2 weeks while cetuximab was intraperitoneally administered at 100 mg/kg twice a week for 2 weeks.

The major and minor axes of tumors were measured with Digimatic caliper (Mitsutoyo), and tumor volumes and relative tumor volumes were calculated according to the following formulae.

Tumor Volume TV=Major axis of tumor(mm)×(Minor axis of tumor)²(mm²)/2

Relative Tumor Volume RTV=Tumor volume on measurement day/Tumor volume on the first administration day

When statistically significant interaction was observed in the combinational use group by two-way ANOVA, a synergistic effect was considered to exist.

As a result, combinational use of E7820 and cetuximab showed a superior anti-tumor effect as compared with the effect obtained with E7820 or cetuximab alone (Table 10).

TABLE 10

| | Relative tumor volume on Day 15 |
|---|---|
| Administered drug | Average ± standard deviation |
| Control (untreated) | 4.1 ± 0.7 |
| E7820 50 mg/kg | 3.3 ± 0.6 |
| Cetuximab 100 mg/kg | 3.2 ± 0.4 |
| E7820 50 mg/kg + | 2.7 ± 0.4 |
| cetuximab 100 mg/kg | |

Table 10 shows anti-tumor effects obtained by the use of E7820 alone, the use of cetuximab alone and the combinational use of E7820 and cetuximab in subcutaneous transplant models of human colon cancer cell line (WiDr). The first day of administration was considered Day 1.

### EXAMPLE 13

### Combinational Use of E7820 and Cetuximab in Subcutaneous Transplant Model (In Vivo) of Human Renal Cancer Cell Line (ACHN)

Human renal cancer cell line ACHN (obtained from Dainippon Pharmaceutical) was cultured in RPMI1640 (containing 10% FBS) in a 5% carbon dioxide incubator at 37° C. to about 80% confluence, and the cells were collected with trypsin-EDTA. Using a phosphate buffer, 1×10⁸ cells/mL suspension was prepared, and 0.1 mL each of the resulting cell suspension was subcutaneously transplanted to a nude mouse at the side of its body. Eight days after the transplantation, E7820 and cetuximab were administered alone or in combination. E7820 was orally administered at 50 mg/kg twice a day for 2 weeks while cetuximab was intraperitoneally administered at 100 mg/kg twice a week for 2 weeks.

The major and minor axes of tumors were measured with Digimatic caliper (Mitsutoyo), and tumor volumes and relative tumor volumes were calculated according to the following formulae.

Tumor Volume TV=Major axis of tumor(mm)×(Minor axis of tumor)² (mm²)/2

Relative Tumor Volume RTV=Tumor volume on measurement day/Tumor volume on the first administration day

When statistically significant interaction was observed in the combinational use group by two-way ANOVA, a synergistic effect was considered to exist.

As a result, combinational use of E7820 and cetuximab showed a superior anti-tumor effect as compared with the effect obtained with E7820 or cetuximab alone (Table 11).

TABLE 11

| | Relative tumor volume on Day 15 |
|---|---|
| Administered drug | Average ± standard deviation |
| Control (untreated) | 2.0 ± 0.2 |
| E7820 50 mg/kg | 1.3 ± 0.2 |
| Cetuximab 100 mg/kg | 1.4 ± 0.1 |
| E7820 50 mg/kg + | 0.9 ± 0.1 |
| cetuximab 100 mg/kg | |

Table 11 shows anti-tumor effects obtained by the use of E7820 alone, the use of cetuximab alone and the combinational use of E7820 and cetuximab in subcutaneous transplant models of human renal cancer cell line (ACHN). The first day of administration was considered Day 1.

From the obtained results, the combination of E7820 and cetuximab was confirmed to provide a pharmaceutical composition and a kit that show a remarkable anti-tumor activity and a method for treating cancer, and thus the pharmaceutical composition, the kit and the method of the invention can be used for treating cancer.

### INDUSTRIAL APPLICABILITY

The present invention provides a pharmaceutical composition and a kit that show a remarkable anti-tumor activity, and a method for treating cancer.

More specifically, the present invention provides a pharmaceutical composition and a kit that show a remarkable anti-tumor activity, and a method for treating cancer, characterized by comprising a sulfonamide compound (i.e., at least one compound selected from: (A) a compound represented by General Formula (I), preferably E7070 or E7820; (B) a compound represented by General Formula (II), preferably LY186641 or LY295501; (C) a compound represented by General Formula (III), preferably LY-ASAP; (D) LY573636; and (E) CQS) in combination with a substance having an EGF inhibitory activity (i.e., at least one substance selected from: (A) an EGF receptor kinase inhibitor, preferably gefitinib, erlotinib, lapatinib, canertinib, pelitinib, AEE-788 or HKI-272; and (B) an anti-EGFR antibody, preferably cetuximab, panitumumab, matuzumab, nimotuzumab, IMC-11F8 or MDX-447). The pharmaceutical composition, the kit and the method of the invention are useful for treating cancer.

1. A pharmaceutical composition comprising a sulfonamide compound in combination with a substance having an EGF inhibitory activity,
wherein the sulfonamide compound is at least one compound selected from the group consisting of:
a compound represented by General Formula (I) 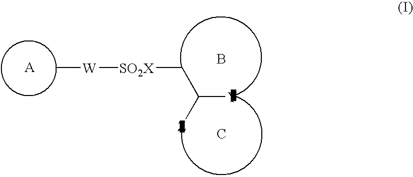
[wherein, ring A represents an optionally substituted monocyclic or bicyclic aromatic ring,
ring B represents an optionally substituted 6-membered cyclic unsaturated hydrocarbon or 6-membered unsaturated heterocycle containing a nitrogen atom as a heteroatom,
ring C represents an optionally substituted 5-membered heterocycle containing one or two nitrogen atoms,
W represents a single bond or —CH═CH—,
X represents —N(R¹)— or an oxygen atom,
Y represents 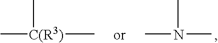
Z represents —N(R²)—,
wherein, R¹, R² and R³ independently represent, identically or differently, a hydrogen atom or a lower alkyl group];
a compound represented by General Formula (II) 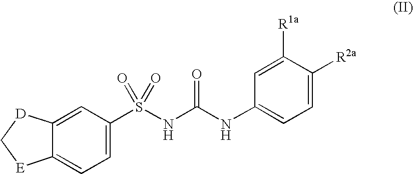
[wherein, E represents —O—, —N(CH₃)—, —CH₂—, —CH₂CH₂— or —CH₂O—, D represents —CH₂— or —O—, R^{1a} represents a hydrogen atom or a halogen atom, and R^{2a} represents a halogen atom or a trifluoromethyl group];
a compound represented by General Formula (III) 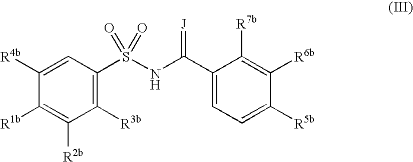
[wherein, J represents —O— or —NH—, R^{1b} represents a hydrogen atom, a halogen atom, an optionally substituted C₁-C₆ alkyl group, an optionally substituted C₁-C₄ alkoxy group, an optionally substituted C₁-C₄ alkylthio group, —CF₃, —OCF₃, —SCF₃, an optionally substituted C₁-C₄ alkoxy carbonyl group, a nitro group, an azido group, —O(SO₂)CH₃, —N(CH₃)₂, a hydroxyl group, a phenyl group, a substituted phenyl group, a pyridinyl group, a thienyl group, a furyl group, a quinolinyl group or a triazole group, R^{2b} represents a hydrogen atom, a halogen atom, a cyano group, —CF₃, an optionally substituted C₁-C₆ alkyl group, an optionally substituted C₁-C₄ alkoxy carbonyl group, an optionally substituted C₁-C₄ alkoxy group, an optionally substituted phenyl group or an optionally substituted quinolinyl group, R^{3b} represents a hydrogen atom or an optionally substituted C₁-C₄ alkoxy group, R^{4b} represents a hydrogen atom or an optionally substituted C₁-C₆ alkyl group (provided that at least one of R^{3b} and R^{4b} is a hydrogen atom), R^{5b} represents a hydrogen atom, a halogen atom, an optionally substituted C₁-C₆ alkyl group, —CF₃ or a nitro group, R^{6b} represents a hydrogen atom, a halogen atom or an optionally substituted C₁-C₆ alkyl group (provided that when R^{6b} is an optionally substituted C₁-C₆ alkyl group, R^{5b} is a hydrogen atom and R^{7b} is a halogen atom), R^{7b} represents a halogen atom, an optionally substituted C₁-C₆ alkyl group or —CF₃ (provided that when either R^{5b} or R^{7b} is an optionally substituted C₁-C₆ alkyl group or when R^{7b} is a halogen atom or an optionally substituted C₁-C₆ alkyl group, either R^{5b} or R^{6b} is a hydrogen atom)];
a compound represented by Formula (IV) 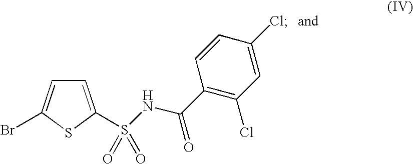
a compound represented by Formula (V) 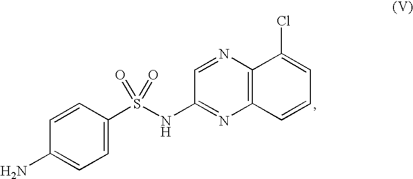
or a pharmacologically acceptable salt thereof, or a solvate thereof.

2. The pharmaceutical composition according to claim 1, wherein the sulfonamide compound is at least one compound selected from the group consisting of:
N-(3-chloro-1H-indole-7-yl)-4-sulfamoylbenzenesulfonamide,
N-(3-cyano-4-methyl-1H-indole-7-yl)-3-cyanobenzenesulfonamide,
N-[[(4-chlorophenyl)amino]carbonyl]-2,3-dihydro-1H-indene-5-sulfonamide,
N-[[(3,4-dichlorophenyl)amino]carbonyl]-2,3-dihydrobenzofuran-5-sulfonamide,
N-(2,4-dichlorobenzoyl)-4-chlorophenylsulfonamide,
N-(2,4-dichlorobenzoyl)-5-bromothiophene-2-sulfonamide, and
2-sulfanylamide-5-chloroquinoxaline,
or a pharmacologically acceptable salt thereof or a solvate thereof.

3. The pharmaceutical composition according to claim 1, wherein the sulfonamide compound is N-(3-cyano-4-methyl-1H-indole-7-yl)-3-cyanobenzene-sulfonamide, a pharmacologically acceptable salt thereof, or a solvate thereof.

4. The pharmaceutical composition according to claim 1, wherein the sulfonamide compound is N-(3-chloro-1H-indole-7-yl)-4-sulfamoylbenzenesulfonamide, a pharmacologically acceptable salt thereof, or a solvate thereof.

5. The pharmaceutical composition according to claim 1, wherein the sulfonamide compound is at least one compound selected from the group consisting of N-[[(3,4-dichlorophenyl)amino]carbonyl]-2,3-dihydrobenzofuran-5-sulfonamide and N-(2,4-dichlorobenzoyl)-5-bromothiophene-2-sulfonamide, or a pharmacologically acceptable salt thereof or a solvate thereof.

6. The pharmaceutical composition according to claim 1, wherein the sulfonamide compound is sodium salt of N-(2,4-dichlorobenzoyl)-5-bromothiophene-2-sulfonamide.

7. The pharmaceutical composition according to claim 1, wherein the substance having an EGF inhibitory activity is an EGF receptor kinase inhibitor.

8. The pharmaceutical composition according to claim 7, wherein the EGF receptor kinase inhibitor is at least one compound selected from the group consisting of:
4-(3-chloro-4-fluorophenylamino)-7-methoxy-6-(3-(4-morpholino)propoxy-quinazoline), 4-(3-ethynylphenylamino)-6,7-bis(2-methoxyethoxy)-quinazoline,
N-[3-chloro-4-[(3-fluorobenzyl)oxy]phenyl]-6-[5-[[[2-(methylsulfonyl)ethyl]-amino]methyl]furan-2-yl]quinazoline-4-amine,
N-[4-[N-(3-chloro-4-fluorophenyl)amino]-7-[3-(4-morpholinyl)propoxy]quinazoline-6-yl]acrylamide,
(2E)-N-[4-[(3-chloro-4-fluorophenyl)amino]-3-cyano-7-ethoxy-6-quinolinyl]-4-(dimethylamino)-2-buteneamide,
[6-[4-[(4-ethylpiperazine-1-yl)methyl]phenyl]-7H-pyrrolo[2,3-d]pyrimidine-4-yl]-((R)-1-phenylethyl)amine, and
(E)-N-{4-[3-chloro-4-(2-pyridinylmethoxy)anilino]-3-cyano-7-ethoxy-6-quinolinyl}-4-(dimethylamino)-2-buteneamide,
or a pharmacologically acceptable salt thereof, or a solvate thereof.

9. The pharmaceutical composition according to claim 7, wherein the EGF receptor kinase inhibitor is gefitinib.

10. The pharmaceutical composition according to claim 7, wherein the EGF receptor kinase inhibitor is erlotinib.

11. The pharmaceutical composition according to claim 1, wherein the substance having an EGF inhibitory activity is an anti-EGFR antibody.

12. The pharmaceutical composition according to claim 11, wherein the anti-EGFR antibody is at least one antibody selected from the group consisting of cetuximab, panitumumab, matuzumab, nimotuzumab, IMC-11F8 and MDX-447.

13. The pharmaceutical composition according to claim 11, wherein the anti-EGFR antibody is cetuximab.

14. (canceled)

15. A kit comprising:
(a) at least one selected from the group consisting of a packaging container, an instruction and a package insert describing the combinational use of a sulfonamide compound and a substance having an EGF inhibitory activity, and
(b) a pharmaceutical composition comprising the sulfonamide compound,
wherein the sulfonamide compound is at least one compound selected from the group consisting of:
a compound represented by General Formula (I) 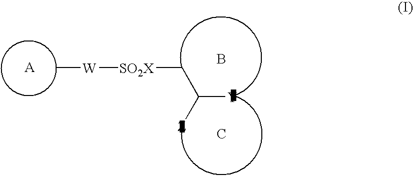
[wherein, ring A represents an optionally substituted monocyclic or bicyclic aromatic ring,
ring B represents an optionally substituted 6-membered cyclic unsaturated hydrocarbon or 6-membered unsaturated heterocycle containing a nitrogen atom as a heteroatom,
ring C represents an optionally substituted 5-membered heterocycle containing one or two nitrogen atoms,
W represents a single bond or —CH═CH—,
X represents —N(R¹)— or an oxygen atom,
Y represents 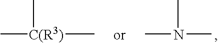
Z represents —N(R²)—,
wherein, R¹, R² and R³ independently represent, identically or differently, a hydrogen atom or a lower alkyl group];
a compound represented by General Formula (II) 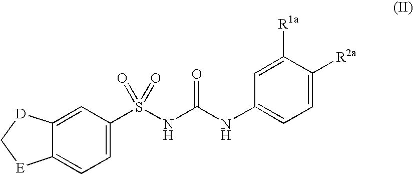
[wherein, E represents —O—, —N(CH₃)—, —CH₂—, —CH₂CH₂— or —CH₂O—, D represents —CH₂— or —O—, R^{1a} represents a hydrogen atom or a halogen atom, and R², represents a halogen atom or a trifluoromethyl group];
a compound represented by General Formula (III) 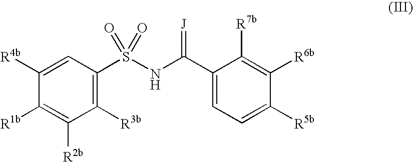
[wherein, J represents —O— or —NH—, R^{1b} represents a hydrogen atom, a halogen atom, an optionally substituted C₁-C₆ alkyl group, an optionally substituted C₁-C₄ alkoxy group, an optionally substituted C₁-C₄ alkylthio group, —CF₃, —OCF₃, —SCF₃, an optionally substituted C₁-C₄ alkoxy carbonyl group, a nitro group, an azido group, —O(SO₂)CH₃, —N(CH₃)₂, a hydroxyl group, a phenyl group, a substituted phenyl group, a pyridinyl group, a thienyl group, a furyl group, a quinolinyl group or a triazole group, R^{2b} represents a hydrogen atom, a halogen atom, a cyano group, —CF₃, an optionally substituted C₁-C₆ alkyl group, an optionally substituted C₁-C₄ alkoxy carbonyl group, an optionally substituted C₁-C₄ alkoxy group, an optionally substituted phenyl group or an optionally substituted quinolinyl group, R^{3b} represents a hydrogen atom or an optionally substituted C₁-C₄ alkoxy group, R^{4b} represents a hydrogen atom or an optionally substituted C₁-C₆ alkyl group (provided that at least one of R^{3b} and R^{4b} is a hydrogen atom), R^{5b} represents a hydrogen atom, a halogen atom, an optionally substituted C₁-C₆ alkyl group, —CF₃ or a nitro group, R^{6b} represents a hydrogen atom, a halogen atom or an optionally substituted C₁-C₆ alkyl group (provided that when R^{6b} is an optionally substituted C₁-C₆ alkyl group, R^{5b} is a hydrogen atom and R^{7b} is a halogen atom), R^{7b} represents a halogen atom, an optionally substituted C₁-C₆ alkyl group or —CF₃ (provided that when either R^{5b} or R^{7b} is an optionally substituted C₁-C₆ alkyl group or when R^{7b} is a halogen atom or an optionally substituted C₁-C₆ alkyl group, either R^{5b} or R^{6b} is a hydrogen atom)];
a compound represented by Formula (IV) 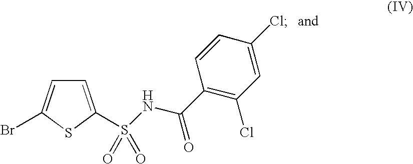
a compound represented by Formula (V) 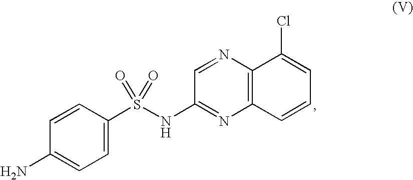
or a pharmacologically acceptable salt thereof, or a solvate thereof.

16. The kit according to claim 15, wherein the sulfonamide compound is at least one compound selected from the group consisting of:
N-(3-chloro-1H-indole-7-yl)-4-sulfamoylbenzenesulfonamide,
N-(3-cyano-4-methyl-1H-indole-7-yl)-3-cyanobenzenesulfonamide,
N-[[(4-chlorophenyl)amino]carbonyl]-2,3-dihydro-1H-indene-5-sulfonamide,
N-[[(3,4-dichlorophenyl)amino]carbonyl]-2,3-dihydrobenzofuran-5-sulfonamide,
N-(2,4-dichlorobenzoyl)-4-chlorophenylsulfonamide,
N-(2,4-dichlorobenzoyl)-5-bromothiophene-2-sulfonamide, and
2-sulfanylamide-5-chloroquinoxaline,
or a pharmacologically acceptable salt thereof, or a solvate thereof.

17. The kit according to claim 15, wherein the sulfonamide compound is N-(3-cyano-4-methyl-1H-indole-7-yl)-3-cyanobenzenesulfonamide, a pharmacologically acceptable salt thereof, or a solvate thereof.

18. The kit according to claim 15, wherein the sulfonamide compound is N-(3-chloro-1H-indole-7-yl)-4-sulfamoylbenzenesulfonamide, a pharmacologically acceptable salt thereof, or a solvate thereof.

19. The kit according to claim 15, wherein the sulfonamide compound is at least one compound selected from the group consisting of N-[[(3,4-dichlorophenyl)amino]carbonyl]-2,3-dihydrobenzofuran-5-sulfonamide and N-(2,4-dichlorobenzoyl)-5-bromothiophene-2-sulfonamide, or a pharmacologically acceptable salt thereof or a solvate thereof.

20. The kit according to claim 15, wherein the sulfonamide compound is sodium salt of N-(2,4-dichlorobenzoyl)-5-bromothiophene-2-sulfonamide.

21. The kit according to any one of claim 15 to 20 claim 15, wherein the substance having an EGF inhibitory activity is an EGF receptor kinase inhibitor.

22. The kit according to claim 21, wherein the EGF receptor kinase inhibitor is at least one compound selected from the group consisting of:
4-(3-chloro-4-fluorophenylamino)-7-methoxy-6-(3-(4-morpholino)propoxy-quinazoline), 4-(3-ethynyl phenylamino)-6,7-bis(2-methoxyethoxy)-quinazoline,
N-[3-chloro-4-[(3-fluorobenzyl)oxy]phenyl]-6-[5-[[[2-(methylsulfonyl)ethyl]amino]-methyl]furan-2-yl]quinazoline-4-amine,
N-[4-[N-(3-chloro-4-fluorophenyl)amino]-7-[3-(4-morpholinyl)propoxy]quinazoline-6-yl]acrylamide,
(2E)-N-[4-[(3-chloro-4-fluorophenyl)amino]-3-cyano-7-ethoxy-6-quinolinyl]-4-(dimethylamino)-2-buteneamide,
[6-[4-[(4-ethylpiperazine-1-yl)methyl]phenyl]-7H-pyrrolo[2,3-d]pyrimidine-4-yl]-((R)-1-phenylethyl)amine, and
(E)-N-{4-[3-chloro-4-(2-pyridinylmethoxy)anilino]-3-cyano-7-ethoxy-6-quinolinyl}-4-(dimethylamino)-2-buteneamide,
or a pharmacologically acceptable salt thereof or a solvate thereof.

23. The kit according to claim 21, wherein the EGF receptor kinase inhibitor is gefitinib.

24. The kit according to claim 21, wherein the EGF receptor kinase inhibitor is erlotinib.

25. The kit according to claim 15, wherein the substance having an EGF inhibitory activity is an anti-EGFR antibody.

26. The kit according to claim 25, wherein the anti-EGFR antibody is at least one antibody selected from the group consisting of cetuximab, panitumumab, matuzumab, nimotuzumab, IMC-11F8 and MDX-447.

27. The kit according to claim 25, wherein the anti-EGFR antibody is cetuximab.

28. (canceled)

29. A kit comprising a set of a formulation comprising a sulfonamide compound and a formulation comprising a substance having an EGF inhibitory activity, wherein the sulfonamide compound is at least one compound selected from the group consisting of:
a compound represented by General Formula (I) 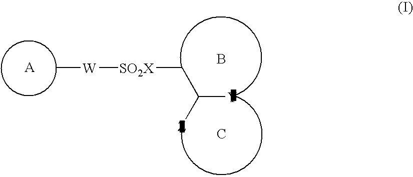
[wherein, ring A represents an optionally substituted monocyclic or bicyclic aromatic ring,
ring B represents an optionally substituted 6-membered cyclic unsaturated hydrocarbon or 6-membered unsaturated heterocycle containing a nitrogen atom as a heteroatom,
ring C represents an optionally substituted 5-membered heterocycle containing one or two nitrogen atoms,
W represents a single bond or —CH═CH—,
X represents —N(R¹)— or an oxygen atom,
Y represents 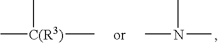
Z represents —N(R²)—,
wherein, R¹, R² and R³ independently represent, identically or differently, a hydrogen atom or a lower alkyl group];
a compound represented by General Formula (II) 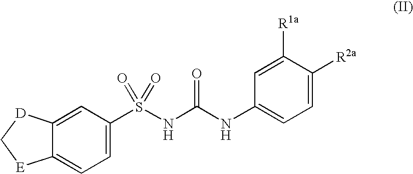
[wherein, E represents —O—, —N(CH₃)—, —CH₂—, —CH₂CH₂— or —CH₂O—, D represents —CH₂— or —O—, R^{1a} represents a hydrogen atom or a halogen atom, and R^{2a} represents a halogen atom or a trifluoromethyl group];
a compound represented by General Formula (III) 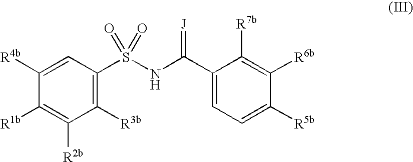
[wherein, J represents —O— or —NH—, R^{1b} represents a hydrogen atom, a halogen atom, an optionally substituted C₁-C₆ alkyl group, an optionally substituted C₁-C₄ alkoxy group, an optionally substituted C₁-C₄ alkylthio group, —CF₃, —OCF₃, —SCF₃, an optionally substituted C₁-C₄ alkoxy carbonyl group, a nitro group, an azido group, —O(SO₂)CH₃, —N(CH₃)₂, a hydroxyl group, a phenyl group, a substituted phenyl group, a pyridinyl group, a thienyl group, a furyl group, a quinolinyl group or a triazole group, R^{2b} represents a hydrogen atom, a halogen atom, a cyano group, —CF₃, an optionally substituted C₁-C₆ alkyl group, an optionally substituted C₁-C₄ alkoxy carbonyl group, an optionally substituted C₁-C₄ alkoxy group, an optionally substituted phenyl group or an optionally substituted quinolinyl group, R^{3b} represents a hydrogen atom or an optionally substituted C₁-C₄ alkoxy group, R^{4b} represents a hydrogen atom or an optionally substituted C₁-C₆ alkyl group (provided that at least one of R^{3b} and R^{4b} is a hydrogen atom), R^{5b} represents a hydrogen atom, a halogen atom, an optionally substituted C₁-C₆ alkyl group, —CF₃ or a nitro group, R^{6b} represents a hydrogen atom, a halogen atom or an optionally substituted C₁-C₆ alkyl group (provided that when R^{6b} is an optionally substituted C₁-C₆ alkyl group, R^{5b} is a hydrogen atom and R^{7b} is a halogen atom), R^{7b} represents a halogen atom, an optionally substituted C₁-C₆ alkyl group or —CF₃ (provided that when either R^{5b} or R^{7b} is an optionally substituted C₁-C₆ alkyl group or when R^{7b} is a halogen atom or an optionally substituted C₁-C₆ alkyl group, either one of R^{5b} or R^{6b} is a hydrogen atom)];
a compound represented by Formula (IV) 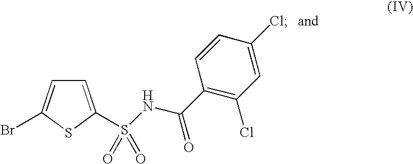
a compound represented by Formula (V) 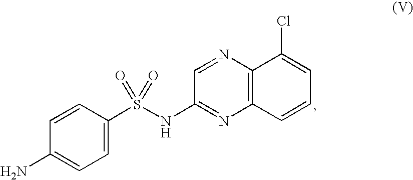
or a pharmacologically acceptable salt thereof or a solvate thereof.

30. The kit according to claim 29, wherein the sulfonamide compound is at least one compound selected from the group consisting of:
N-(3-chloro-1H-indole-7-yl)-4-sulfamoylbenzenesulfonamide,
N-(3-cyano-4-methyl-1H-indole-7-yl)-3-cyanobenzenesulfonamide,
N-[[(4-chlorophenyl)amino]carbonyl]-2,3-dihydro-1H-indene-5-sulfonamide,
N-[[(3,4-dichlorophenyl)amino]carbonyl]-2,3-dihydrobenzofuran-5-sulfonamide,
N-(2,4-dichlorobenzoyl)-4-chlorophenylsulfonamide,
N-(2,4-dichlorobenzoyl)-5-bromothiophene-2-sulfonamide, and
2-sulfanylamide-5-chloroquinoxaline,
or a pharmacologically acceptable salt thereof or a solvate thereof.

31. The kit according to claim 29, wherein the sulfonamide compound is N-(3-cyano-4-methyl-1H-indole-7-yl)-3-cyanobenzenesulfonamide, a pharmacologically acceptable salt thereof, or a solvate thereof.

32. The kit according to claim 29, wherein the sulfonamide compound is N-(3-chloro-1H-indole-7-yl)-4-sulfamoylbenzenesulfonamide, a pharmacologically acceptable salt thereof or a solvate thereof.

33. The kit according to claim 29, wherein the sulfonamide compound is at least one compound selected from the group consisting of N-[[(3,4-dichlorophenyl)-amino]carbonyl]-2,3-dihydrobenzofuran-5-sulfonamide and N-(2,4-dichlorobenzoyl)-5-bromothiophene-2-sulfonamide, or a pharmacologically acceptable salt thereof, or a solvate thereof.

34. The kit according to claim 29, wherein the sulfonamide compound is sodium salt of N-(2,4-dichlorobenzoyl)-5-bromothiophene-2-sulfonamide.

35. The kit according to claim 29, wherein the substance having an EGF inhibitory activity is an EGF receptor kinase inhibitor.

36. The kit according to claim 35, wherein the EGF receptor kinase inhibitor is at least one compound selected from the group consisting of:
4-(3-chloro-4-fluorophenylamino)-7-methoxy-6-(3-(4-morpholino)propoxy-quinazoline);
4-(3-ethynylphenylamino)-6,7-bis(2-methoxyethoxy)-quinazoline;
N-[3-chloro-4-[(3-fluorobenzyl)oxy]phenyl]-6-[5-[[[2-(methylsulfonyl)ethyl]-amino]methyl]furan-2-yl]quinazoline-4-amine;
N-[4-[N-(3-chloro-4-fluorophenyl)amino]-7-[3-(4-morpholinyl)propoxy]quinazoline-6-yl]acrylamide;
(2E)-N-[4-[(3-chloro-4-fluorophenyl)amino]-3-cyano-7-ethoxy-6-quinolinyl]-4-(dimethylamino)-2-buteneamide;
[6-[4-[(4-ethylpiperazine-1-yl)methyl]phenyl]-7H-pyrrolo[2,3-d]pyrimidine-4-yl]-((R)-1-phenylethyl)amine; and
(E)-N-{4-[3-chloro-4-(2-pyridinylmethoxy)anilino]-3-cyano-7-ethoxy-6-quinolinyl}-4-(dimethylamino)-2-buteneamide,
or a pharmacologically acceptable salt thereof or a solvate thereof.

37. The kit according to claim 35, wherein the EGF receptor kinase inhibitor is gefitinib.

38. The kit according to claim 35, wherein the EGF receptor kinase inhibitor is erlotinib.

39. The kit according to claim 29, wherein the substance having an EGF inhibitory activity is an anti-EGFR antibody.

40. The kit according to claim 39, wherein the anti-EGFR antibody is at least one antibody selected from the group consisting of cetuximab, panitumumab, matuzumab, nimotuzumab, IMC-11F8 and MDX-447.

41. The kit according to claim 39, wherein the anti-EGFR antibody is cetuximab.

42. (canceled)

43. A method for producing a pharmaceutical composition in combination comprising combining a sulfonamide compound with a substance having an EGF inhibitory activity, wherein the sulfonamide compound is at least one compound selected from the group consisting of:
a compound represented by General Formula (I) 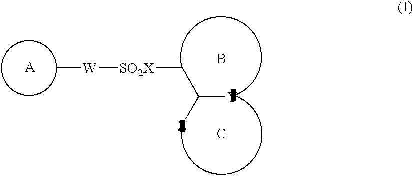
[wherein, ring A represents an optionally substituted monocyclic or bicyclic aromatic ring,
ring B represents an optionally substituted 6-membered cyclic unsaturated hydrocarbon or 6-membered unsaturated heterocycle containing a nitrogen atom as a heteroatom,
ring C represents an optionally substituted 5-membered heterocycle containing one or two nitrogen atoms,
W represents a single bond or —CH═CH—,
X represents —N(R¹)— or an oxygen atom,
Y represents 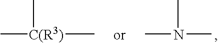
Z represents —N(R²)—,
wherein, R¹, R² and R³ independently represent, identically or differently, a hydrogen atom or a lower alkyl group];
a compound represented by General Formula (II) 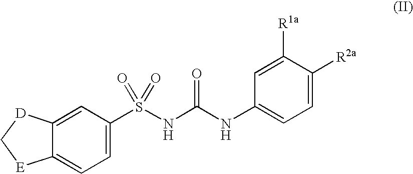
[wherein, E represents —O—, —N(CH₃)—, —CH₂—, —CH₂CH₂— or —CH₂O—, D represents —CH₂— or —O—, R^{1a} represents a hydrogen atom or a halogen atom, and R^{2a} represents a halogen atom or a trifluoromethyl group];
a compound represented by General Formula (III) 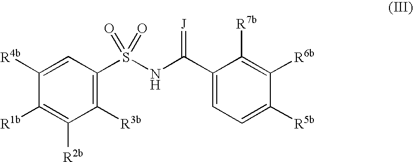
[wherein, J represents —O— or —NH—, R^{1b} represents a hydrogen atom, a halogen atom, an optionally substituted C₁-C₆ alkyl group, an optionally substituted C₁-C₄ alkoxy group, an optionally substituted C₁-C₄ alkylthio group, —CF₃, —OCF₃, —SCF₃, an optionally substituted C₁-C₄ alkoxy carbonyl group, a nitro group, an azido group, —O(SO₂)CH₃, —N(CH₃)₂, a hydroxyl group, a phenyl group, a substituted phenyl group, a pyridinyl group, a thienyl group, a furyl group, a quinolinyl group or a triazole group, R^{2b} represents a hydrogen atom, a halogen atom, a cyano group, —CF₃, an optionally substituted C₁-C₆ alkyl group, an optionally substituted C₁-C₄ alkoxy carbonyl group, an optionally substituted C₁-C₄ alkoxy group, an optionally substituted phenyl group or an optionally substituted quinolinyl group, R^{3b} represents a hydrogen atom or an optionally substituted C₁-C₄ alkoxy group, R^{4b} represents a hydrogen atom or an optionally substituted C₁-C₆ alkyl group (provided that at least one of R^{3b} and R^{4b} is a hydrogen atom), R^{5b} represents a hydrogen atom, a halogen atom, an optionally substituted C₁-C₆ alkyl group, —CF₃ or a nitro group, R^{6b} represents a hydrogen atom, a halogen atom or an optionally substituted C₁-C₆ alkyl group (provided that when R^{6b} is an optionally substituted C₁-C₆ alkyl group, R^{5b} is a hydrogen atom and R^{7b} is a halogen atom), R^{7b} represents a halogen atom, an optionally substituted C₁-C₆ alkyl group or —CF₃ (provided that when either R^{5b} or R^{7b} is an optionally substituted C₁-C₆ alkyl group or when R^{7b} is a halogen atom or an optionally substituted C₁-C₆ alkyl group, either R^{5b} or R^{6b} is a hydrogen atom)];
a compound represented by Formula (IV) 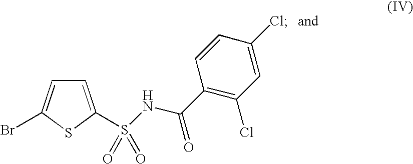
a compound represented by Formula (V) 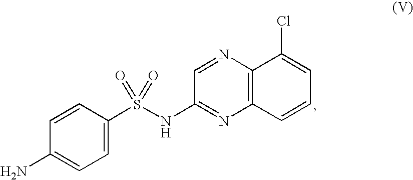
or a pharmacologically acceptable salt thereof or a solvate thereof.

44. The method according to claim 43, wherein the sulfonamide compound is at least one compound selected from the group consisting of:
N-(3-chloro-1H-indole-7-yl)-4-sulfamoylbenzenesulfonamide;
N-(3-cyano-4-methyl-1H-indole-7-yl)-3-cyanobenzenesulfonamide;
N-[[(4-chlorophenyl)amino]carbonyl]-2,3-dihydro-1H-indene-5-sulfonamide;
N-[[(3,4-dichlorophenyl)amino]carbonyl]-2,3-dihydrobenzofuran-5-sulfonamide;
N-(2,4-dichlorobenzoyl)-4-chlorophenylsulfonamide;
N-(2,4-dichlorobenzoyl)-5-bromothiophene-2-sulfonamide; and
2-sulfanylamide-5-chloroquinoxaline,
or a pharmacologically acceptable salt thereof or a solvate thereof.

45. The method according to claim 43, wherein the sulfonamide compound is N-(3-cyano-4-methyl-1H-indole-7-yl)-3-cyanobenzenesulfonamide, a pharmacologically acceptable salt thereof, or a solvate thereof.

46. The method according to claim 43, wherein the sulfonamide compound is N-(3-chloro-1H-indole-7-yl)-4-sulfamoylbenzenesulfonamide, a pharmacologically acceptable salt thereof, or a solvate thereof.

47. The method according to claim 43, wherein the sulfonamide compound is at least one compound selected from the group consisting of N-[[(3,4-dichlorophenyl)amino]carbonyl]-2,3-dihydrobenzofuran-5-sulfonamide and N-(2,4-dichlorobenzoyl)-5-bromothiophene-2-sulfonamide, or a pharmacologically acceptable salt thereof or a solvate thereof.

48. The method according to claim 43, wherein the sulfonamide compound is sodium salt of N-(2,4-dichlorobenzoyl)-5-bromothiophene-2-sulfonamide.

49. The method according to claim 43, wherein the substance having an EGF inhibitory activity is an EGF receptor kinase inhibitor.

50. The method according to claim 49, wherein the EGF receptor kinase inhibitor is at least one compound selected from the group consisting of:
4-(3-chloro-4-fluorophenylamino)-7-methoxy-6-(3-(4-morpholino)propoxy-quinazoline);
4-(3-ethynylphenylamino)-6,7-bis(2-methoxyethoxy)-quinazoline;
N-[3-chloro-4-[(3-fluorobenzyl)oxy]phenyl]-6-[5-[[[2-(methylsulfonyl)ethyl]-amino]methyl]furan-2-yl]quinazoline-4-amine;
N-[4-[N-(3-chloro-4-fluorophenyl)amino]-7-[3-(4-morpholinyl)propoxy]quinazoline-6-yl]acrylamide;
(2E)-N-[4-[(3-chloro-4-fluorophenyl)amino]-3-cyano-7-ethoxy-6-quinolinyl]-4-(dimethylamino)-2-buteneamide;
[6-[4-[(4-ethyl piperazine-1-yl)methyl]phenyl]-7H-pyrrolo[2,3-d]pyrimidine-4-yl]-((R)-1-phenylethyl)amine; and
(E)-N-{4-[3-chloro-4-(2-pyridinylmethoxy)anilino]-3-cyano-7-ethoxy-6-quinolinyl}-4-(dimethylamino)-2-buteneamide,
or a pharmacologically acceptable salt thereof or a solvate thereof.

51. The method according to claim 49, wherein the EGF receptor kinase inhibitor is gefitinib.

52. The method according to claim 49, wherein the EGF receptor kinase inhibitor is erlotinib.

53. The method according to claim 43, wherein the substance having an EGF inhibitory activity is an anti-EGFR antibody.

54. The method according to claim 53, wherein the anti-EGFR antibody is at least one antibody selected from the group consisting of cetuximab, panitumumab, matuzumab, nimotuzumab, IMC-11F8 and MDX-447.

55. The method according to claim 53, wherein the anti-EGFR antibody is cetuximab.

56. (canceled)

57. A method for treating cancer comprising administering a sulfonamide compound and a substance having an EGF inhibitory activity to a patient, wherein the sulfonamide compound is at least one compound selected from the group consisting of:
a compound represented by General Formula (I) 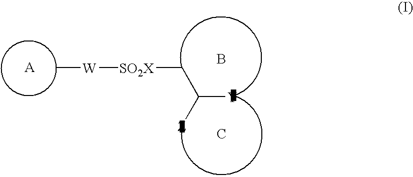
[wherein, ring A represents an optionally substituted monocyclic or bicyclic aromatic ring,
ring B represents an optionally substituted 6-membered cyclic unsaturated hydrocarbon or 6-membered unsaturated heterocycle containing a nitrogen atom as a heteroatom,
ring C represents an optionally substituted 5-membered heterocycle containing one or two nitrogen atoms,
W represents a single bond or —CH═CH—,
X represents —N(R¹)— or an oxygen atom,
Y represents 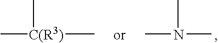
Z represents —N(R²)—,
wherein, R¹, R² and R³ independently represent, identically or differently, a hydrogen atom or a lower alkyl group];
a compound represented by General Formula (II) 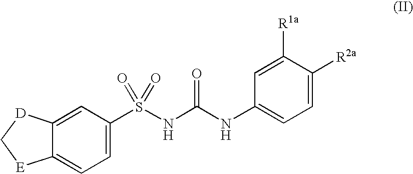
[wherein, E represents —O—, —N(CH₃)—, —CH₂—, —CH₂CH₂— or —CH₂O—, D represents —CH₂— or —O—, R^{1a} represents a hydrogen atom or a halogen atom, and R^{2a} represents a halogen atom or a trifluoromethyl group];
a compound represented by General Formula (III) 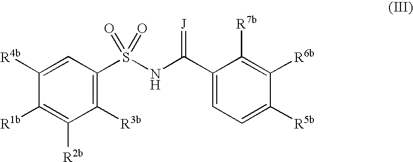
[wherein, J represents —O— or —NH—, R^{1b} represents a hydrogen atom, a halogen atom, an optionally substituted C₁-C₆ alkyl group, an optionally substituted C₁-C₄ alkoxy group, an optionally substituted C₁-C₄ alkylthio group, —CF₃, —OCF₃, —SCF₃, an optionally substituted C₁-C₄ alkoxy carbonyl group, a nitro group, an azido group, —O(SO₂)CH₃, —N(CH₃)₂, a hydroxyl group, a phenyl group, a substituted phenyl group, a pyridinyl group, a thienyl group, a furyl group, a quinolinyl group or a triazole group, R^{2b} represents a hydrogen atom, a halogen atom, a cyano group, —CF₃, an optionally substituted C₁-C₆ alkyl group, an optionally substituted C₁-C₄ alkoxy carbonyl group, an optionally substituted C₁-C₄ alkoxy group, an optionally substituted phenyl group or an optionally substituted quinolinyl group, R^{3b} represents a hydrogen atom or an optionally substituted C₁-C₄ alkoxy group, R^{4b} represents a hydrogen atom or an optionally substituted C₁-C₆ alkyl group (provided that at least one of R^{3b} and R^{4b} is a hydrogen atom), R^{5b} represents a hydrogen atom, a halogen atom, an optionally substituted C₁-C₆ alkyl group, —CF₃ or a nitro group, R^{6b} represents a hydrogen atom, a halogen atom or an optionally substituted C₁-C₆ alkyl group (provided that when R^{6b} is an optionally substituted C₁-C₆ alkyl group, R^{5b} is a hydrogen atom and R^{7b} is a halogen atom), R^{7b} represents a halogen atom, an optionally substituted C₁-C₆ alkyl group or —CF₃ (provided that when either R^{5b} or R^{7b} is an optionally substituted C₁-C₆ alkyl group or when R^{7b} is a halogen atom or an optionally substituted C₁-C₆ alkyl group, either R^{5b} or R^{6b} is a hydrogen atom)];
a compound represented by Formula (IV) 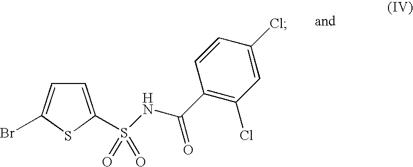
a compound represented by Formula (V) 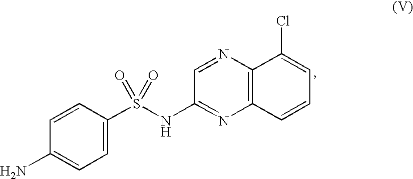
or a pharmacologically acceptable salt thereof or a solvate thereof.

58. The method according to claim 57, wherein the sulfonamide compound is at least one compound selected from the group consisting of:
N-(3-chloro-1H-indole-7-yl)-4-sulfamoylbenzenesulfonamide;
N-(3-cyano-4-methyl-1H-indole-7-yl)-3-cyanobenzenesulfonamide;
N-[[(4-chlorophenyl)amino]carbonyl]-2,3-dihydro-1H-indene-5-sulfonamide;
N-[[(3,4-dichlorophenyl)amino]carbonyl]-2,3-dihydrobenzofuran-5-sulfonamide;
N-(2,4-dichlorobenzoyl)-4-chlorophenylsulfonamide;
N-(2,4-dichlorobenzoyl)-5-bromothiophene-2-sulfonamide; and
2-sulfanylamide-5-chloroquinoxaline,
or a pharmacologically acceptable salt thereof or a solvate thereof.

59. The method according to claim 57, wherein the sulfonamide compound is N-(3-cyano-4-methyl-1H-indole-7-yl)-3-cyanobenzenesulfonamide, a pharmacologically acceptable salt thereof or a solvate thereof.

60. The method according to claim 57, wherein the sulfonamide compound is N-(3-chloro-1H-indole-7-yl)-4-sulfamoylbenzenesulfonamide, a pharmacologically acceptable salt thereof or a solvate thereof.

61. The method according to claim 57, wherein the sulfonamide compound is at least one compound selected from the group consisting of N-[[(3,4-dichlorophenyl)amino]carbonyl]-2,3-dihydrobenzofuran-5-sulfonamide and N-(2,4-dichlorobenzoyl)-5-bromothiophene-2-sulfonamide, or a pharmacologically acceptable salt thereof or a solvate thereof.

62. The method according to claim 57, wherein the sulfonamide compound is sodium salt of N-(2,4-dichlorobenzoyl)-5-bromothiophene-2-sulfonamide.

63. The method according to claim 57, wherein the substance having an EGF inhibitory activity is an EGF receptor kinase inhibitor.

64. The method according to claim 63, wherein the EGF receptor kinase inhibitor is at least one compound selected from the group consisting of:
4-(3-chloro-4-fluorophenylamino)-7-methoxy-6-(3-(4-morpholino)propoxy-quinazoline);
4-(3-ethynylphenylamino)-6,7-bis(2-methoxyethoxy)-quinazoline;
N-[3-chloro-4-[(3-fluorobenzyl)oxy]phenyl]-6-[5-[[[2-(methylsulfonyl)ethyl]-amino]methyl]furan-2-yl]quinazoline-4-amine;
N-[4-[N-(3-chloro-4-fluorophenyl)amino]-7-[3-(4-morpholinyl)propoxy]quinazoline-6-yl]acrylamide;
(2E)-N-[4-[(3-chloro-4-fluorophenyl)amino]-3-cyano-7-ethoxy-6-quinolinyl]-4-(dimethylamino)-2-buteneamide;
[6-[4-[(4-ethylpiperazine-1-yl)methyl]phenyl]-7H-pyrrolo[2,3-d]pyrimidine-4-yl]-((R)-1-phenylethyl)amine; and
(E)-N-{4-[3-chloro-4-(2-pyridinylmethoxy)anilino]-3-cyano-7-ethoxy-6-quinolinyl}-4-(dimethylamino)-2-buteneamide,
or a pharmacologically acceptable salt thereof or a solvate thereof.

65. The method according to claim 63, wherein the EGF receptor kinase inhibitor is gefitinib.

66. The method according to claim 63, wherein the EGF receptor kinase inhibitor is erlotinib.

67. The method according to claim 57, wherein the substance having an EGF inhibitory activity is an anti-EGFR antibody.

68. The method according to claim 67, wherein the anti-EGFR antibody is at least one antibody selected from the group consisting of cetuximab, panitumumab, matuzumab, nimotuzumab, IMC-11F8 and MDX-447.

69. The method according to claim 67, wherein the anti-EGFR antibody is cetuximab.

70. A pharmaceutical composition comprising a sulfonamide compound for administering to a patient in combination with a substance having an EGF inhibitory activity, wherein the sulfonamide compound is at least one compound selected from the group consisting of:
a compound represented by General Formula (I) 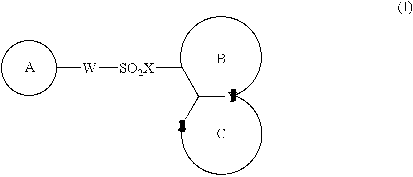
[wherein, ring A represents an optionally substituted monocyclic or bicyclic aromatic ring,
ring B represents an optionally substituted 6-membered cyclic unsaturated hydrocarbon or 6-membered unsaturated heterocycle containing a nitrogen atom as a heteroatom,
ring C represents an optionally substituted 5-membered heterocycle containing one or two nitrogen atoms,
W represents a single bond or —CH═CH—,
X represents —N(R¹)— or an oxygen atom,
Y represents 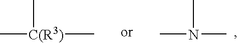
Z represents —N(R²)—,
wherein, R¹, R² and R³ independently represent, identically or differently, a hydrogen atom or a lower alkyl group];
a compound represented by General Formula (II) 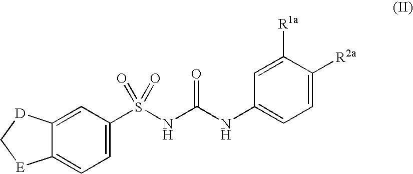
[wherein, E represents —O—, —N(CH₃)—, —CH₂—, —CH₂CH₂— or —CH₂O—, D represents —CH₂— or —O—, R^{1a} represents a hydrogen atom or a halogen atom, and R^{2a} represents a halogen atom or a trifluoromethyl group];
a compound represented by General Formula (III) 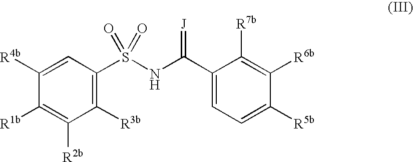
[wherein, J represents —O— or —NH—, R^{1b} represents a hydrogen atom, a halogen atom, an optionally substituted C₁-C₆ alkyl group, an optionally substituted C₁-C₄ alkoxy group, an optionally substituted C₁-C₄ alkylthio group, —CF₃, —OCF₃, —SCF₃, an optionally substituted C₁-C₄ alkoxy carbonyl group, a nitro group, an azido group, —O(SO₂)CH₃, —N(CH₃)₂, a hydroxyl group, a phenyl group, a substituted phenyl group, a pyridinyl group, a thienyl group, a furyl group, a quinolinyl group or a triazole group, R^{2b} represents a hydrogen atom, a halogen atom, a cyano group, —CF₃, an optionally substituted C₁-C₆ alkyl group, an optionally substituted C₁-C₄ alkoxy carbonyl group, an optionally substituted C₁-C₄ alkoxy group, an optionally substituted phenyl group or an optionally substituted quinolinyl group, R^{3b} represents a hydrogen atom or an optionally substituted C₁-C₄ alkoxy group, R^{4b} represents a hydrogen atom or an optionally substituted C₁-C₆ alkyl group (provided that at least one of R^{3b} and R^{4b} is a hydrogen atom), R^{5b} represents a hydrogen atom, a halogen atom, an optionally substituted C₁-C₆ alkyl group, —CF₃ or a nitro group, R^{6b} represents a hydrogen atom, a halogen atom or an optionally substituted C₁-C₆ alkyl group (provided that when R^{6b} is an optionally substituted C₁-C₆ alkyl group, R^{5b} is a hydrogen atom and R^{7b} is a halogen atom), R^{7b} represents a halogen atom, an optionally substituted C₁-C₆ alkyl group or —CF₃ (provided that when either R^{5b} or R^{7b} is an optionally substituted C₁-C₆ alkyl group or when R^{7b} is a halogen atom or an optionally substituted C₁-C₆ alkyl group, either R^{5b} or R^{6b} is a hydrogen atom)];
a compound represented by Formula (IV) 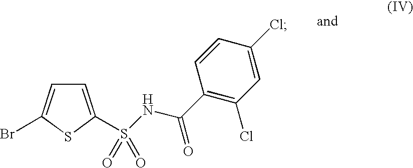
a compound represented by Formula (V) 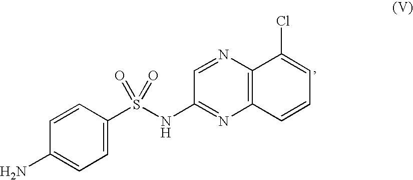
or a pharmacologically acceptable salt thereof or a solvate thereof.

71. The pharmaceutical composition according to claim 70, wherein the sulfonamide compound is at least one compound selected from the group consisting of:
N-(3-chloro-1H-indole-7-yl)-4-sulfamoylbenzenesulfonamide,
N-(3-cyano-4-methyl-1H-indole-7-yl)-3-cyanobenzenesulfonamide,
N-[[(4-chlorophenyl)amino]carbonyl]-2,3-dihydro-1H-indene-5-sulfonamide,
N-[[(3,4-dichlorophenyl)amino]carbonyl]-2,3-dihydrobenzofuran-5-sulfonamide,
N-(2,4-dichlorobenzoyl)-4-chlorophenylsulfonamide,
N-(2,4-dichlorobenzoyl)-5-bromothiophene-2-sulfonamide, and
2-sulfanylamide-5-chloroquinoxaline,
or a pharmacologically acceptable salt thereof or a solvate thereof.

72. The pharmaceutical composition according to claim 70, wherein the sulfonamide compound is N-(3-cyano-4-methyl-1H-indole-7-yl)-3-cyanobenzene-sulfonamide, a pharmacologically acceptable salt thereof or a solvate thereof.

73. The pharmaceutical composition according to claim 70, wherein the sulfonamide compound is N-(3-chloro-1H-indole-7-yl)-4-sulfamoylbenzenesulfonamide, a pharmacologically acceptable salt thereof or a solvate thereof.

74. The pharmaceutical composition according to claim 70, wherein the sulfonamide compound is at least one compound selected from the group consisting of: N-[[(3,4-dichlorophenyl)amino]carbonyl]-2,3-dihydrobenzofuran-5-sulfonamide; and N-(2,4-dichlorobenzoyl)-5-bromothiophene-2-sulfonamide, or a pharmacologically acceptable salt thereof or a solvate thereof.

75. The pharmaceutical composition according to claim 70, wherein the sulfonamide compound is sodium salt of N-(2,4-dichlorobenzoyl)-5-bromothiophene-2-sulfonamide.

76. The pharmaceutical composition according to claim 70, wherein the substance having an EGF inhibitory activity is an EGF receptor kinase inhibitor.

77. The pharmaceutical composition according to claim 76, wherein the EGF receptor kinase inhibitor is at least one compound selected from the group consisting of:
4-(3-chloro-4-fluorophenylamino)-7-methoxy-6-(3-(4-morpholino)propoxy-quinazoline);
4-(3-ethynylphenylamino)-6,7-bis(2-methoxyethoxy)-quinazoline;
N-[3-chloro-4-[(3-fluorobenzyl)oxy]phenyl]-6-[5-[[[2-(methylsulfonyl)ethyl]-amino]methyl]furan-2-yl]quinazoline-4-amine;
N-[4-[N-(3-chloro-4-fluorophenyl)amino]-7-[3-(4-morpholinyl)propoxy]quinazoline-6-yl]acrylamide;
(2E)-N-[4-[(3-chloro-4-fluorophenyl)amino]-3-cyano-7-ethoxy-6-quinolinyl]-4-(dimethylamino)-2-buteneamide;
[6-[4-[(4-ethylpiperazine-1-yl)methyl]phenyl]-7H-pyrrolo[2,3-d]pyrimidine-4-yl]-((R)-1-phenylethyl)amine; and
(E)-N-{4-[3-chloro-4-(2-pyridinylmethoxy)anilino]-3-cyano-7-ethoxy-6-quinolinyl}-4-(dimethylamino)-2-buteneamide,
or a pharmacologically acceptable salt thereof or a solvate thereof.

78. The pharmaceutical composition according to claim 76, wherein the EGF receptor kinase inhibitor is gefitinib.

79. The pharmaceutical composition according to claim 76, wherein the EGF receptor kinase inhibitor is erlotinib.

80. The pharmaceutical composition according to claim 70, wherein the substance having an EGF inhibitory activity is an anti-EGFR antibody.

81. The pharmaceutical composition according to claim 80, wherein the anti-EGFR antibody is at least one antibody selected from the group consisting of cetuximab, panitumumab, matuzumab, nimotuzumab, IM C-11F8 and MDX-447.

82. The pharmaceutical composition according to claim 80, wherein the anti-EGFR antibody is cetuximab.

83. (canceled)
